(19) United States
(12) Patent Application Publication
(10) Pub. No.: US 2009/0163516 A1
(43) Pub. Date: Jun. 25, 2009

(54) CARBOXAMIDES FOR CONTROLLING UNDESIRED MICRO-ORGANISMS IN PLANT PROTECTION

(75) Inventors: Ralf Dunkel, Lyon (FR); Hans-Ludwig Elbe, Wuppertal (DE); Jörg Nico Greul, Leichlingen (DE); Herbert Gayer, Monheim (DE); Peter Dahmen, Neuss (DE); Ulrike Wachendorff-Neumann, Neuwied (DE); Arnd Voerste, Koln (DE)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(73) Assignee: Bayer Cropscience AG, Monheim (DE)

(21) Appl. No.: 12/097,492

(22) PCT Filed: Dec. 5, 2006
(86) PCT No.: PCT/EP2006/011651

§ 371 Date: Sep. 29, 2008

(30) Foreign Application Priority Data
Dec. 17, 2005 (DE) 102005060449.8

(51) Int. Cl. A01N 43/54; C07D 239/42; C07D 417/12; A01N 43/78; A01N 43/56; C07D 403/12; C07D 409/12

(52) U.S. Cl. 514/256; 544/329; 544/328

(57) ABSTRACT

Novel carboxamides of the formula (I)

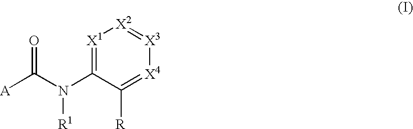

There are also provided a plurality of processes for preparing these compounds and their use for controlling unwanted microorganisms, and also novel intermediates and their preparation.

The present invention relates to novel carboxamides, to a plurality of processes for their preparation and to their use for controlling harmful microorganisms in crop protection and in the protection of materials.

It is already known that numerous carboxamides have fungicidal properties (cf., for example, WO 03/070705, EP-A 0 545 099 and JP-A 9-132567). The activity of the compounds described in these publications is good; however, it is sometimes unsatisfactory.

This invention now provides novel carboxamides of the formula (I)

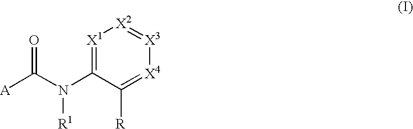

in which
R represents phenyl which is optionally mono- to pentasubstituted by W¹ or represents the grouping

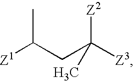

W¹ represents halogen, cyano, nitro, C₁-C₆-alkyl, C₁-C₄-alkoxy, C₁-C₄-alkylthio, C₁-C₄-alkylsulphonyl, C₂-C₆-alkenyl, C₃-C₆-cycloalkyl or represents C₁-C₆-haloalkyl, C₁-C₆-haloalkoxy, C₁-C₆-haloalkylthio or C₁-C₆-haloalkylsulphonyl having in each case 1 to 13 halogen atoms, or represents —C(Q²)=N-Q³ in which
   Q² represents hydrogen, hydroxyl, C₁-C₄-alkyl, C₁-C₄-haloalkyl having 1 to 9 halogen atoms or C₃-C₆-cycloalkyl,
   Q³ represents hydroxyl, amino, methylamino, phenyl, benzyl or represents in each case optionally halogen-, cyano-, hydroxyl-, C₁-C₄-alkoxy-, C₁-C₄-alkylthio-, C₁-C₄-alkylamino, di(C₁-C₄-alkyl)amino or phenyl-substituted C₁-C₄-alkyl or C₁-C₄-alkoxy, or represents C₂-C₄-alkenyloxy or C₂-C₄-Alkynyloxy,
Z¹ represents hydrogen or methyl,
Z² represents hydrogen or methyl,
Z³ represents methyl or ethyl,
R¹ represents hydrogen, C₁-C₈-alkyl, C₁-C₆-alkylsulphinyl, C₁-C₆-alkylsulphonyl, C₁-C₄-alkoxy-C₁-C₄-alkyl, C₃-C₈-cycloalkyl; C₁-C₆-haloalkyl, C₁-C₄-haloalkylthio, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkoxy)carbonyl, (C₁-C₆-haloalkylthio)carbonyl, (halo-C₁-C₄-alkoxy-C₁-C₄-alkyl)carbonyl, (C₃-C₆-haloalkenyloxy)carbonyl, (C₃-C₆-haloalkynyloxy)carbonyl, (C₃-C₈-halocycloalkyl)carbonyl having in each case 1 to 9 fluorine, chlorine and/or bromine atoms; or —CH₂—C≡C—R^{1-A}, —CH₂—CH═CH—R^{1-A}, —CH═C═CH—R^{1-A}, —C(═O)C(═O)R², —CONR³R⁴ or —CH₂NR⁵R⁶,
R^{1-A} represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,
R² represents hydrogen, C₁-C₈-alkyl, C₁-C₈-alkoxy, C₁-C₄-alkoxy-C₁-C₄-alkyl, C₃-C₈-cycloalkyl; C₁-C₆-haloalkyl, C₁-C₆-haloalkoxy, halo-C₁-C₄-alkoxy-C₁-C₄-alkyl, C₃-C₈-halocycloalkyl having in each case 1 to 9 fluorine, chlorine and/or bromine atoms,
R³ and R⁴ independently of one another each represent hydrogen, C₁-C₈-alkyl, C₁-C₄-alkoxy-C₁-C₄-alkyl, C₃-C₈-cycloalkyl; C₁-C₈-haloalkyl, halo-C₁-C₄-alkoxy-C₁-C₄-alkyl, C₃-C₈-halocycloalkyl having in each case 1 to 9 fluorine, chlorine and/or bromine atoms,
R³ and R⁴ furthermore together with the nitrogen atom to which they are attached form a saturated heterocycle which is optionally mono- or polysubstituted by identical or different substituents from a group consisting of halogen and C₁-C₄-alkyl and which has 5 to 8 ring atoms, where the heterocycle may contain 1 or 2 further non-adjacent heteroatoms from the group consisting of oxygen, sulphur and NR⁷,
R⁵ and R⁶ independently of one another represent hydrogen, C₁-C₈-alkyl, C₃-C₈-cycloalkyl; C₁-C₈-haloalkyl, C₃-C₈-halocycloalkyl having in each case 1 to 9 fluorine, chlorine and/or bromine atoms,
R⁵ and R⁶ furthermore together with the nitrogen atom to which they are attached form a saturated heterocycle which is optionally mono- or polysubstituted by identical or different substituents from a group consisting of halogen and C₁-C₄-alkyl and which has 5 to 8 ring atoms, where the heterocycle may contain 1 or 2 further non-adjacent heteroatoms from the group consisting of oxygen, sulphur and NR⁷,
R⁷ represents hydrogen or C₁-C₆-alkyl,
X¹, X², X³ and X⁴ independently of one another represent N or CR⁸, with the proviso that at least one of these radicals represents N,
R⁸ represents hydrogen, fluorine, chlorine, methyl, isopropyl, methylthio or trifluoromethyl,
A represents one of the radicals A1 to A18 below

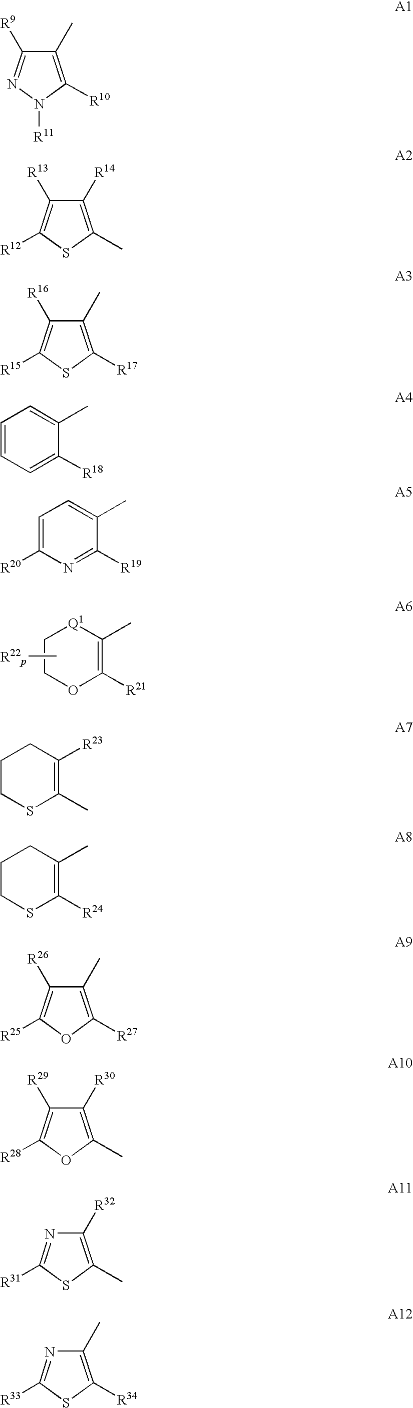

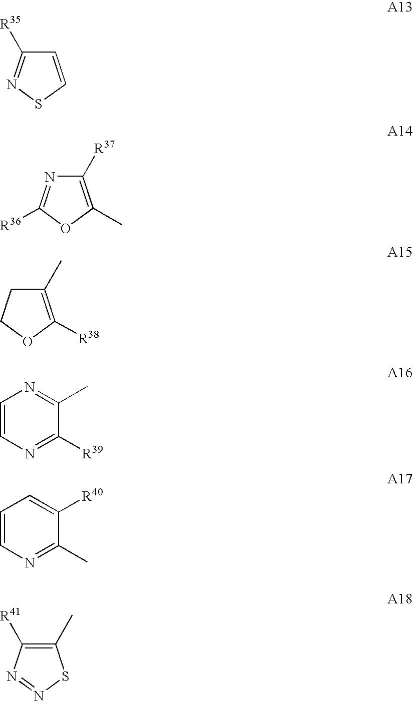

R⁹ represents hydrogen, cyano, halogen, nitro, C₁-C₄-alkyl, C₁-C₄-alkoxy, C₁-C₄-alkylthio, C₃-C₆-cycloalkyl, C₁-C₄-haloalkyl, C₁-C₄-haloalkoxy or C₁-C₄-haloalkylthio having in each case 1 to 5 halogen atoms, aminocarbonyl or aminocarbonyl-C₁-C₄-alkyl,
R¹⁰ represents hydrogen, halogen, cyano, C₁-C₄-alkyl, C₁-C₄-alkoxy or C₁-C₄-alkylthio,
R¹¹ represents hydrogen, C₁-C₄-alkyl, hydroxy-C₁-C₄-alkyl, C₂-C₆-alkenyl, C₃-C₆-cycloalkyl, C₁-C₄-alkylthio-C₁-C₄-alkyl, C₁-C₄-alkoxy-C₁-C₄-alkyl, C₁-C₄-haloalkyl, C₁-C₄-haloalkylthio-C₁-C₄-alkyl, C₁-C₄-haloalkoxy-C₁-C₄-alkyl having in each case 1 to 5 halogen atoms, phenyl, (C₁-C₄-alkyl)carbonyl, (C₁-C₄-alkoxy)carbonyl, (C₁-C₄-alkylthio)carbonyl, (C₁-C₄-alkoxy-C₁-C₄-alkyl)carbonyl; (C₁-C₄-haloalkyl)carbonyl, (C₁-C₄-haloalkoxy)carbonyl, (C₁-C₄-haloalkylthio)carbonyl, (halo-C₁-C₄-alkoxy-C₁-C₄-alkyl)carbonyl having in each case 1 to 9 halogen atoms,
R¹² and R¹³ independently of one another represent hydrogen, halogen, C₁-C₄-alkyl or C₁-C₄-haloalkyl having 1 to 5 halogen atoms,
R¹⁴ represents halogen, cyano or C₁-C₄-alkyl, or C₁-C₄-haloalkyl or C₁-C₄-haloalkoxy having in each case 1 to 5 halogen atoms,
R¹⁵ and R¹⁶ independently of one another represent hydrogen, halogen, C₁-C₄-alkyl or C₁-C₄-haloalkyl having 1 to 5 halogen atoms,
R¹⁷ represents hydrogen, C₁-C₄-alkyl or C₁-C₄-haloalkyl having 1 to 5 halogen atoms,
R¹⁸ represents hydrogen, halogen, hydroxyl, cyano, C₁-C₆-alkyl, C₁-C₄-haloalkyl, C₁-C₄-haloalkoxy or C₁-C₄-haloalkylthio having in each case 1 to 5 halogen atoms,
R¹⁹ represents halogen, hydroxyl, cyano, C₁-C₄-alkyl, C₁-C₄-alkoxy, C₁-C₄-alkylthio, C₁-C₄-haloalkyl, C₁-C₄-haloalkylthio or C₁-C₄-haloalkoxy having in each case 1 to 5 halogen atoms,
R²⁰ represents hydrogen, halogen, cyano, C₁-C₄-alkyl, C₁-C₄-alkoxy, C₁-C₄-alkylthio, C₁-C₄-haloalkyl, C₁-C₄-haloalkoxy having in each case 1 to 5 halogen atoms, C₁-C₄-alkylsulphinyl or C₁-C₄-alkylsulphonyl,
R²¹ represents C₁-C₄-alkyl or C₁-C₄-haloalkyl having 1 to 5 halogen atoms,
R²² represents C₁-C₄-alkyl,
Q¹ represents S (sulphur), SO, SO₂ or CH₂,
p represents 0, 1 or 2, where R²² represents identical or different radicals if p represents 2,
R²³ represents C₁-C₄-alkyl or C₁-C₄-haloalkyl having to 5 halogen atoms,
R²⁴ represents C₁-C₄-alkyl or C₁-C₄-haloalkyl having 1 to 5 halogen atoms,
R²⁵ and R²⁶ independently of one another represent hydrogen, halogen, amino, C₁-C₄-alkyl or C₁-C₄-haloalkyl having 1 to 5 halogen atoms,
R²⁷ represents hydrogen, C₁-C₄-alkyl or C₁-C₄-haloalkyl having 1 to 5 halogen atoms,
R²⁸ and R²⁹ independently of one another represent hydrogen, halogen, amino, nitro, C₁-C₄-alkyl or C₁-C₄-haloalkyl having 1 to 5 halogen atoms,
R³⁰ represents hydrogen, halogen, C₁-C₄-alkyl or C₁-C₄-haloalkyl having 1 to 5 halogen atoms,
R³¹ represents hydrogen, halogen, amino, C₁-C₄-alkylamino, di-(C₁-C₄-alkyl)amino, cyano, C₁-C₄-alkyl or C₁-C₄-haloalkyl having 1 to 5 halogen atoms,
R³² represents halogen, C₁-C₄-alkyl or C₁-C₄-haloalkyl having 1 to 5 halogen atoms,
R³³ represents hydrogen, halogen, amino, C₁-C₄-alkylamino, di-(C₁-C₄-alkyl)amino, cyano, C₁-C₄-alkyl or C₁-C₄-haloalkyl having 1 to 5 halogen atoms,
R³⁴ represents halogen, C₁-C₄-alkyl or C₁-C₄-haloalkyl having 1 to 5 halogen atoms,
R³⁵ represents halogen, C₁-C₄-alkyl or C₁-C₄-haloalkyl having 1 to 5 halogen atoms,
R³⁶ represents hydrogen or C₁-C₄-alkyl,
R³⁷ represents halogen or C₁-C₄-alkyl,
R³⁸ represents C₁-C₄-alkyl or C₁-C₄-haloalkyl having 1 to 5 halogen atoms,
R³⁹ represents hydrogen, halogen, C₁-C₄-alkyl or C₁-C₄-haloalkyl having 1 to 5 halogen atoms,
R⁴⁰ represents halogen, hydroxyl, C₁-C₄-alkyl, C₁-C₄-alkoxy, C₁-C₄-alkylthio, C₁-C₄-haloalkyl, C₁-C₄-haloalkylthio or C₁-C₄-haloalkoxy having in each case 1 to 5 halogen atoms,
R⁴¹ represents C₁-C₄-alkyl.

Furthermore, it has been found that carboxamides of the formula (I) are obtained when

(a) carbonyl halides of the formula (II)

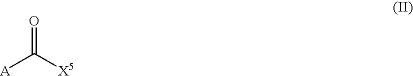

in which
A is as defined above,
X⁵ represents halogen or hydroxyl,
are reacted with aniline derivatives of the formula (III)

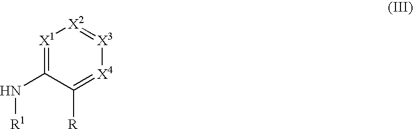

in which
R, R¹, X¹, X², X³ and X⁴ are as defined above,
if appropriate in the presence of a coupling agent, if appropriate in the presence of an acid binder and if appropriate in the presence of a diluent,
   or
   (b) carboxamides of the formula (I-a)

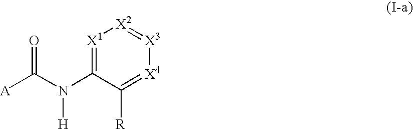

in which R, X¹, X², X³, X⁴ and A are as defined above
are reacted with halides of the formula (IV)

R^{1-B}-Hal (I)
in which
R^{1-B} represents C₁-C₈-alkyl, C₁-C₆-alkylsulphinyl, C₁-C₆-alkylsulphonyl, C₁-C₄-alkoxy-C₁-C₄-alkyl, C₃-C₈-cycloalkyl; C₁-C₆-haloalkyl, C₁-C₄-haloalkylthio, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo(C₁-C₃-alkyl)carbonyl-C₁-C₃-alkyl, halo(C₁-C₃-alkoxy)carbonyl-C₁-C₃-alkyl having in each case 1 to 13 fluorine, chlorine and/or bromine atoms;
(C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkoxy)carbonyl, (C₁-C₆-haloalkylthio)carbonyl, (halo-C₁-C₄-alkoxy-C₁-C₄-alkyl)carbonyl, (C₃-C₆-haloalkenyloxy)carbonyl, (C₃-C₆-haloalkynyloxy)carbonyl, (C₃-C₈-halocycloalkyl)carbonyl having in each case 1 to 9 fluorine, chlorine and/or bromine atoms; or —CH₂—C≡C—R^{1-A}, —CH₂—CH═CH—R^{1-A}, —CH═C═CH—R^{1-A}, —C(═O)C(═O)R², —CONR³R⁴ or —CH₂NR⁵R⁶, R^{1-A}, R², R³, R⁴, R⁵ and R⁶ are as defined above,
Hal represents chlorine, bromine or iodine,
in the presence of a base and in the presence of a diluent.

Finally, it has been found that the novel carboxamides of the formula (I) have very good microbicidal properties and can be used for controlling unwanted microorganisms both in crop protection and in the protection of materials.

If appropriate, the compounds according to the invention can be present as mixtures of different possible isomeric forms, in particular of stereoisomers, such as, for example, E and Z, threo and erythro, and also optical isomers, and, if appropriate, also of tautomers. What is claimed are both the E and the Z isomers, and also the threo and erythro, and the optical isomers, any mixtures of these isomers, and the possible tautomeric forms.

The formula (I) provides a general definition of the biphenylthiazolecarboxamides according to the invention. Preferred radical definitions of the formulae mentioned above and below are listed below. These definitions apply to the end products of the formula (I) and likewise to all intermediates.
R preferably represents monosubstituted phenyl, where the substituents are selected from the list W¹.
R also preferably represents phenyl which is disubstituted by identical or different substituents, where the substituents are selected from the list W¹.
R also preferably represents phenyl which is trisubstituted by identical or different substituents, where the substituents are selected from the list W¹.
R particularly preferably represents phenyl which is monosubstituted in the 4-position, where the substituents are selected from the list W¹.
R particularly preferably represents phenyl which is disubstituted by identical or different substituents in the 3,4-position, where the substituents are selected from the list W¹.
R particularly preferably represents phenyl which is disubstituted by identical or different substituents in the 2,4-position, where the substituents are selected from the list W¹.
R particularly preferably represents phenyl which is disubstituted by identical or different substituents in the 3,5-position, where the substituents are selected from the list W¹.
R particularly preferably represents phenyl which is trisubstituted by identical or different substituents in the 2,4,6-position, where the substituents are selected from the list W¹.
R also preferably represents one of the groupings Z1 to Z8 below

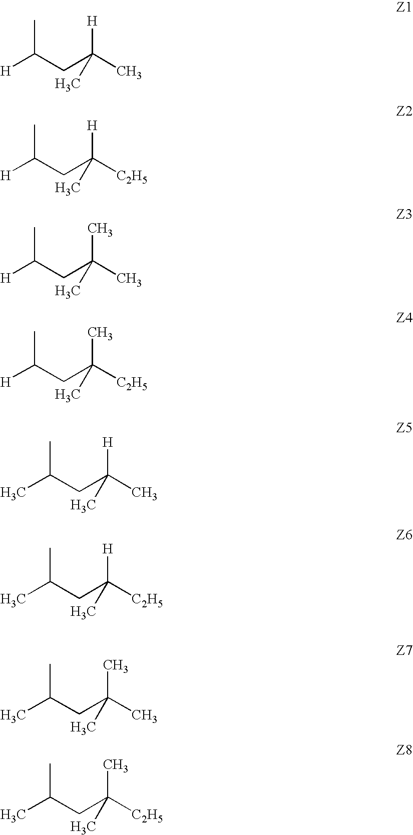

R also particularly preferably represents Z1, Z3, Z5 or Z7.
R also very particularly preferably represents Z1.
R also very particularly preferably represents Z3.
R also very particularly preferably represents Z5.
R also very particularly preferably represents Z7.
W¹ preferably represents fluorine, chlorine, bromine, iodine, cyano, nitro, C₁-C₄-alkyl, C₁-C₄-alkoxy, C₁-C₄-alkylthio, C₁-C₄-alkylsulphonyl, C₂-C₄-alkenyl, C₃-C₆-cycloalkyl or represents C₁-C₄-haloalkyl, C₁-C₄-haloalkoxy, C₁-C₄-haloalkylthio or C₁-C₄-haloalkylsulphonyl having in each case 1 to 9 fluorine, chlorine and/or bromine atoms or represents —C(Q²)=N-Q³, in which
   Q² represents hydrogen, methyl, ethyl, trifluoromethyl or cyclopropyl and
   Q³ represents hydroxyl, methoxy, ethoxy, propoxy or isopropoxy.
W¹ particularly preferably represents fluorine, chlorine, bromine, iodine, methyl, ethyl, n-, isopropyl, n-, i-, s-, t-butyl, methoxy, ethoxy, n-, isopropoxy, n-, i-, s-, t-butoxy, methylthio, ethylthio, n-, isopropylthio, n-, i-, s-, t-butylthio, methylsulphonyl, ethylsulphonyl, n-, isopropylsulphonyl, n-, i-, s-, t-butylsulphonyl, allyl, cyclopropyl, cyclobutyl, cyclopentyl, cyclohexyl, monofluoromethyl, monofluoroethyl, difluoromethyl, trifluoromethyl, difluorochloromethyl, trichloromethyl, dichloromethyl, monofluoromethoxy, monofluoroethoxy, difluoromethoxy, trifluoromethoxy, difluorochloromethoxy, trichloromethoxy, dichloromethoxy, monofluoromethylthio, monofluoroethylthio, difluoromethylthio, trifluoromethylthio, difluorochloromethylthio, trichloromethylthio, dichloromethylthio, monofluoromethylsulphonyl, monofluoroethylsulphonyl, difluoromethylsulphonyl, trifluoromethylsulphonyl, difluorochloromethylsulphonyl, trichloromethylsulphonyl, dichloromethylsulphonyl, —CH═N—OH, —CH═N—OCH₃, —CH═N—OC₂H₅, —CH═N—O(n-C₃H₇), —CH═N—O(i-C₃H₇), —C(CH₃)═N—OH, —C(CH₃)═N—OCH₃, —C(CH₃)═N—OC₂H₅, —C(CH₃)═N—O(n-C₃H₇), —C(CH₃)═N—O(i-C₃H₇), —C(C₂H₅)═N—OH, —C(C₂H₅)═N—OCH₃, —C(C₂H₅)═N—OC₂H₅, —C(C₂H₅)═N—O(n-C₃H₇), —C(C₂H₅)═N—O(i-C₃H₇), —C(CF₃)═N—OH, —C(CF₃)═N—OCH₃, —C(CF₃)═N—OC₂H₅, —C(CF₃)═N—O(n-C₃H₇), —C(CF₃)═N—O(i-C₃H₇), —C(cyclopropyl)═N—OH, —C(cyclopropyl)=N—OCH₃, —C(cyclopropyl)=N—OC₂H₅, —C(cyclopropyl)=N—O(n-C₃H₇), —C(cyclopropyl)=N—O(i-C₃H₇).
W¹ very particularly preferably represents fluorine, chlorine, bromine, methyl, methoxy, methylthio, methylsulphonyl, difluoromethyl, trifluoromethyl, trichloromethyl, dichloromethyl, trifluoromethoxy, trichloromethoxy, trifluoromethylthio, trichloromethylthio, trifluoromethylsulphonyl, —C(CH₃)═N—OCH₃, —C(CH₃)═N—O(i-C₃H₇), —C(cyclopropyl)=N—OCH₃.
R¹ preferably represents hydrogen, C₁-C₆-alkyl, C₁-C₄-alkylsulphinyl, C₁-C₄-alkylsulphonyl, C₁-C₃-alkoxy-C₁-C₃-alkyl, C₃-C₆-cycloalkyl; C₁-C₄-haloalkyl, C₁-C₄-haloalkylthio, C₁-C₄-haloalkylsulphinyl, C₁-C₄-haloalkylsulphonyl, halo-C₁-C₃-alkoxy-C₁-C₃-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms;
   (C₁-C₆-alkyl)carbonyl, (C₁-C₄-alkoxy)carbonyl, (C₁-C₄-alkylthio)carbonyl, (C₁-C₃-alkoxy-C₁-C₃-alkyl)carbonyl, (C₃-C₄-alkenyloxy)carbonyl, (C₃-C₄-alkynyloxy)carbonyl, (C₃-C₆-cycloalkyl)carbonyl; (C₁-C₄-haloalkyl)carbonyl, (C₁-C₄-haloalkoxy)carbonyl, (C₁-C₄-haloalkylthio)carbonyl, (halo-C₁-C₃-alkoxy-C₁-C₃-alkyl)carbonyl, (C₃-C₄-haloalkenyloxy)carbonyl, (C₃-C₄-haloalkynyloxy)carbonyl, (C₃-C₆-halocycloalkyl)carbonyl having in each case 1 to 9 fluorine, chlorine and/or bromine atoms; or —CH₂—C≡C—R^{1-A}, —CH₂—CH═CH—R^{1-A}, —CH═C═CH—R^{1-A}, —C(═O)C(═O)R², —CONR³R⁴ or —CH₂NR⁵R⁶.
R¹ particularly preferably represents hydrogen, methyl, ethyl, n- or isopropyl, n-, iso-, sec- or tert-butyl, pentyl or hexyl, methylsulphinyl, ethylsulphinyl, n- or isopropylsulphinyl, n-, iso-, sec- or tert-butylsulphinyl, methylsulphonyl, ethylsulphonyl, n- or isopropylsulphonyl, n-, iso-, sec- or tert-butylsulphonyl, methoxymethyl, methoxyethyl, ethoxymethyl, ethoxyethyl, cyclopropyl, cyclopentyl, cyclohexyl, trifluoromethyl, trichloromethyl, trifluoroethyl, difluoromethylthio, difluorochloromethylthio, trifluoromethylthio, trifluoromethylsulphinyl, trifluoromethylsulphonyl, trifluoromethoxymethyl; formyl, —CH₂—CHO, —(CH₂)₂—CHO, —CH₂—CO—CH₃, —CH₂—CO—CH₂CH₃, —CH₂—CO—CH(CH₃)₂, —(CH₂)₂—CO—CH₃, —(CH₂)₂—CO—CH₂CH₃, —(CH₂)₂—CO—CH(CH₃)₂, —CH₂—CO₂CH₃, —CH₂—CO₂CH₂CH₃, —CH₂—CO₂CH(CH₃)₂, —(CH₂)₂—CO₂CH₃, —(CH₂)₂—CO₂CH₂CH₃, —(CH₂)₂—CO₂CH(CH₃)₂, —CH₂—CO—CF₃, —CH₂—CO—CCl₃, —CH₂—CO—CH₂CF₃, —CH₂—CO—CH₂CCl₃, —(CH₂)₂—CO—CH₂CF₃, —(CH₂)₂—CO—CH₂CCl₃, —CH₂—CO₂CH₂CF₃, —CH₂—CO₂CF₂CF₃, —CH₂—CO₂CH₂CCl₃, —CH₂—CO₂CCl₂CCl₃, —(CH₂)₂—CO₂CH₂CF₃, —(CH₂)₂—CO₂CF₂CF₃, —(CH₂)₂—CO₂CH₂CCl₃, —(CH₂)₂—CO₂CCl₂CCl₃;
   methylcarbonyl, ethylcarbonyl, n-propylcarbonyl, isopropylcarbonyl, tert-butylcarbonyl, methoxycarbonyl, ethoxycarbonyl, isopropoxycarbonyl, tert-butoxycarbonyl, methylthiocarbonyl, ethylthiocarbonyl, isopropylthiocarbonyl, tert-butylthiocarbonyl, methoxymethylcarbonyl, ethoxymethylcarbonyl, cyclopropylcarbonyl; trifluoromethylcarbonyl, trifluoromethoxycarbonyl, trifluoromethylthiocarbonyl, or —CH₂—C≡C—R^{1-A}, —CH₂—CH═CH—R^{1-A}, —CH═C═CH—R^{1-A}, —C(═O)C(═O)R², —CONR³R⁴ or —CH₂NR⁵R⁶.
R¹ very particularly preferably represents hydrogen, methyl, methoxymethyl, methoxymethylcarbonyl, ethoxymethylcarbonyl, formyl, —CH₂—C≡CH, —CH₂—CH═CH₂, —CH═C═CH₂, —CH₂—CHO, —(CH₂)₂—CHO, —CH₂—CO—CH₃, —CH₂—CO—CH₂CH₃, —CH₂—CO—CH(CH₃)₂, —C(═O)CHO, —C(═O)C(═O)CH₃, —C(═O)C(═O)CH₂OCH₃, —C(═O)CO₂CH₃, —C(═O)CO₂CH₂CH₃.
R^{1-A} preferably represents hydrogen, C₁-C₄-alkyl, C₁-C₄-haloalkyl, C₂-C₄-alkenyl, C₂-C₄-alkynyl, C₃-C₆-cycloalkyl, (C₁-C₄-alkoxy)carbonyl, or cyano.
R^{1-A} particularly preferably represents hydrogen, methyl or ethyl.
R² preferably represents hydrogen, C₁-C₆-alkyl, C₁-C₄-alkoxy, C₁-C₃-alkoxy-C₁-C₃-alkyl, C₃-C₆-cycloalkyl; C₁-C₄-haloalkyl, C₁-C₄-haloalkoxy, halo-C₁-C₃-alkoxy-C₁-C₃-alkyl, C₃-C₆-halocycloalkyl having in each case 1 to 9 fluorine, chlorine and/or bromine atoms.
R² particularly preferably represents hydrogen, methyl, ethyl, n- or isopropyl, tert-butyl, methoxy, ethoxy, n- or isopropoxy, tert-butoxy, methoxymethyl, cyclopropyl; trifluoromethyl, trifluoromethoxy.
R³ and R⁴ independently of one another preferably represent hydrogen, C₁-C₆-alkyl, C₁-C₃-alkoxy-C₁-C₃-alkyl, C₃-C₆-cycloalkyl; C₁-C₄-haloalkyl, halo-C₁-C₃-alkoxy-C₁-C₃-alkyl, C₃-C₆-halocycloalkyl having in each case 1 to 9 fluorine, chlorine and/or bromine atoms.
R³ and R⁴ furthermore together with the nitrogen atom to which they are attached preferably represent a saturated heterocycle which is optionally mono- to tetrasubstituted by identical or different substituents from the group consisting of halogen and C₁-C₄-alkyl and which has 5 or 6 ring atoms, where the heterocycle may contain 1 or 2 further non-adjacent heteroatoms from the group consisting of oxygen, sulphur and NR⁷.
R³ and R⁴ independently of one another particularly preferably represent hydrogen, methyl, ethyl, n- or isopropyl, n-, iso-, sec- or tert-butyl, methoxymethyl, methoxyethyl, ethoxymethyl, ethoxyethyl, cyclopropyl, cyclopentyl, cyclohexyl; trifluoromethyl, trichloromethyl, trifluoroethyl, trifluoromethoxymethyl.
R³ and R⁴ furthermore together with the nitrogen atom to which they are attached particularly preferably form a saturated heterocycle from the group consisting of morpholine, thiomorpholine and piperazine, which is optionally mono- to tetrasubstituted by identical or different substituents from the group consisting of fluorine, chlorine, bromine and methyl, where the piperazine may be substituted on the second nitrogen atom by R⁷.
R⁵ and R⁶ independently of one another preferably represent hydrogen, C₁-C₆-alkyl, C₃-C₆-cycloalkyl; C₁-C₄-haloalkyl, C₃-C₆-halocycloalkyl having in each case 1 to 9 fluorine, chlorine and/or bromine atoms.
R⁵ and R⁶ furthermore together with the nitrogen atom to which they are attached preferably form a saturated heterocycle which is optionally mono- to tetrasubstituted by identical or different substituents from the group consisting of halogen and C₁-C₄-alkyl and which has 5 or 6 ring atoms, where the heterocycle may contain 1 or 2 further non-adjacent heteroatoms from the group consisting of oxygen, sulphur and NR⁷.
R⁵ and R⁶ independently of one another particularly preferably represent hydrogen, methyl, ethyl, n- or isopropyl, n-, iso-, sec- or tert-butyl, methoxymethyl, methoxyethyl, ethoxymethyl, ethoxyethyl, cyclopropyl, cyclopentyl, cyclohexyl; trifluoromethyl, trichloromethyl, trifluoroethyl, trifluoromethoxymethyl.
R⁵ and R⁶ furthermore together with the nitrogen atom to which they are attached particularly preferably form a saturated heterocycle from the group consisting of morpholine, thiomorpholine and piperazine, which is optionally mono- to tetrasubstituted by identical or different substituents from the group consisting of fluorine, chlorine, bromine and methyl, where the piperazine may be substituted on the second nitrogen atom by R⁷.
R⁷ preferably represents hydrogen or C₁-C₄-alkyl.
R⁷ particularly preferably represents hydrogen, methyl, ethyl, n- or isopropyl, n-, iso-, sec- or tert-butyl.

Preferably, X¹, X² and X³ each represent CR⁸ and X⁴ represents N.

Furthermore, preferably X¹, X² and X⁴ each represent CR⁸ and X³ represents N.

Furthermore, preferably X¹, X³ and X⁴ each represent CR⁸ and X² represents N.

Furthermore, preferably X², X³ and X⁴ each represent CR⁸ and X¹ represents N.

Preferably, X² and X⁴ each represent CR⁸ and X¹ and X³ each represent N.

Furthermore, preferably X² and X³ each represent CR⁸ and X¹ and X⁴ each represent N.

Furthermore, preferably X³ and X⁴ each represent CR⁸ and X¹ and X² each represent N.

Furthermore, preferably X¹ and X⁴ each represent CR⁸ and X² and X³ each represent N.

Particularly preferably, X² and X⁴ each represent CR⁸ and X¹ and X³ each represent N.

Very particularly preferably, X² and X⁴ each represent CH and X¹ and X³ each represent N.
R⁸ preferably represents hydrogen, fluorine, chlorine, methyl or trifluoromethyl.
R⁸ particularly preferably represents hydrogen.
R⁸ furthermore particularly preferably represents fluorine.
R⁸ furthermore particularly preferably represents chlorine.
R⁸ furthermore particularly preferably represents methyl.
R⁸ furthermore particularly preferably represents trifluoromethyl.
A preferably represents one of the radicals A1, A2, A3, A4, A5, A6, A9, A10, A11, A12 or A17.
A particularly preferably represents one of the radicals A1, A2, A4, A5, A6, A9, A11, A16, A17.
A very particularly preferably represents the radical A1.
A furthermore very particularly preferably represents the radical A2.
A furthermore very particularly preferably represents the radical A4.
A furthermore very particularly preferably represents the radical A5.
A furthermore very particularly preferably represents the radical A6.
A furthermore very particularly preferably represents the radical A9.
A furthermore very particularly preferably represents the radical A11.
A furthermore very particularly preferably represents the radical A16.
A furthermore very particularly preferably represents the radical A17.
R⁹ preferably represents hydrogen, cyano, fluorine, chlorine, bromine, iodine, methyl, ethyl, isopropyl, methoxy, ethoxy, methylthio, ethylthio, cyclopropyl, C₁-C₂-haloalkyl, C₁-C₂-haloalkoxy having in each case 1 to 5 fluorine, chlorine and/or bromine atoms, trifluoromethylthio, difluoromethylthio, aminocarbonyl, aminocarbonylmethyl or aminocarbonylethyl.
R⁹ particularly preferably represents hydrogen, fluorine, chlorine, bromine, iodine, methyl, ethyl, isopropyl, monofluoromethyl, monofluoroethyl, difluoromethyl, trifluoromethyl, difluorochloromethyl, trichloromethyl, dichloromethyl, cyclopropyl, methoxy, ethoxy, trifluoromethoxy, trichloromethoxy, methylthio, ethylthio, trifluoromethylthio or difluoromethylthio.
R⁹ very particularly preferably represents hydrogen, fluorine, chlorine, bromine, iodine, methyl, isopropyl, monofluoromethyl, monofluoroethyl, difluoromethyl, trifluoromethyl, difluoro-chloromethyl or trichloromethyl.

R⁹ especially preferably represents methyl, difluoromethyl, trifluoromethyl or 1-fluoroethyl.
R¹⁰ preferably represents hydrogen, fluorine, chlorine, bromine, iodine, methyl, ethyl, methoxy, ethoxy, methylthio or ethylthio.
R¹⁰ particularly preferably represents hydrogen, fluorine, chlorine, bromine, iodine or methyl.
R¹⁰ very particularly preferably represents hydrogen, fluorine, chlorine or methyl.
R¹¹ preferably represents hydrogen, methyl, ethyl, n-propyl, isopropyl, C₁-C₂-haloalkyl having 1 to 5 fluorine, chlorine and/or bromine atoms, hydroxymethyl, hydroxyethyl, cyclopropyl, cyclopentyl, cyclohexyl, phenyl, methylcarbonyl, n-propylcarbonyl, tert-butylcarbonyl, methoxycarbonyl, isopropoxycarbonyl, tert-butoxycarbonyl, methoxymethylcarbonyl.
R¹¹ particularly preferably represents hydrogen, methyl, ethyl, isopropyl, trifluoromethyl, difluoromethyl, hydroxymethyl, hydroxyethyl, phenyl, methylcarbonyl, isopropoxycarbonyl, tert-butoxycarbonyl, methoxymethylcarbonyl.
R¹¹ very particularly preferably represents hydrogen, methyl, trifluoromethyl, phenyl or methylcarbonyl.
R¹¹ especially preferably represents methyl.
R¹² and R¹³ independently of one another preferably represent hydrogen, fluorine, chlorine, bromine, methyl, ethyl or C₁-C₂-haloalkyl having 1 to 5 fluorine, chlorine and/or bromine atoms.
R¹² and R¹³ independently of one another particularly preferably represent hydrogen, fluorine, chlorine, bromine, methyl, ethyl, difluoromethyl, trifluoromethyl, difluorochloromethyl or trichloromethyl.
R¹² and R¹³ independently of one another ver particularly preferably represent hydrogen, fluorine, chlorine, bromine, methyl, ethyl, difluoromethyl, trifluoromethyl or trichloromethyl.
R¹² and R¹³ especially preferably each represent hydrogen.
R¹⁴ preferably represents fluorine, chlorine, bromine, cyano, methyl, ethyl, C₁-C₂-haloalkyl or C₁-C₂-haloalkoxy having in each case 1 to 5 fluorine, chlorine and/or bromine atoms.
R¹⁴ particularly preferably represents fluorine, chlorine, bromine, cyano, methyl, trifluoromethyl, trifluoromethoxy, difluoromethoxy, difluorochloromethoxy or trichloromethoxy.
R¹⁴ very particularly preferably represents fluorine, chlorine, bromine, iodine, methyl, trifluoromethyl or trifluoromethoxy.
R¹⁴ especially preferably represents methyl or trifluoromethyl.
R¹⁵ and R¹⁶ independently of one another preferably represent hydrogen, fluorine, chlorine, bromine, methyl, ethyl or C₁-C₂-haloalkyl having 1 to 5 fluorine, chlorine and/or bromine atoms.
R¹⁵ and R¹⁶ independently of one another particularly preferably represent hydrogen, fluorine, chlorine, bromine, methyl, ethyl, difluoromethyl, trifluoromethyl, difluorochloromethyl or trichloromethyl.
R¹⁵ and R¹⁶ independently of one another very particularly preferably represent hydrogen, fluorine, chlorine, bromine, methyl, ethyl, difluoromethyl, trifluoromethyl or trichloromethyl.
R¹⁵ and R¹⁶ especially preferably each represent hydrogen.
R¹⁷ preferably represents hydrogen, methyl, ethyl or C₁-C₂-haloalkyl having 1 to 5 fluorine, chlorine and/or bromine atoms.
R¹⁷ particularly preferably represents hydrogen, methyl or trifluoromethyl.
R¹⁷ very particularly preferably represents methyl or trifluoromethyl.
R¹⁸ preferably represents hydrogen, fluorine, chlorine, bromine, iodine, hydroxyl, cyano, C₁-C₄-alkyl, C₁-C₂-haloalkyl, C₁-C₂-haloalkoxy or C₁-C₂-haloalkylthio having in each case 1 to 5 fluorine, chlorine and/or bromine atoms.
R¹⁸ particularly preferably represents hydrogen, fluorine, chlorine, bromine, iodine, hydroxyl, cyano, methyl, ethyl, n-propyl, isopropyl, n-butyl, iso-butyl, sec-butyl, tert-butyl, difluoromethyl, trifluoromethyl, difluorochloromethyl, trichloromethyl, trifluoromethoxy, difluoromethoxy, difluorochloromethoxy, trichloromethoxy, trifluoromethylthio, difluoromethylthio, difluorochloromethylthio or trichloromethylthio.
R¹⁸ very particularly preferably represents hydrogen, fluorine, chlorine, bromine, iodine, methyl, difluoromethyl, trifluoromethyl or trichloromethyl.
R¹⁸ especially preferably represents iodine, methyl, difluoromethyl or trifluoromethyl.
R¹⁹ preferably represents fluorine, chlorine, bromine, iodine, hydroxyl, cyano, C₁-C₄-alkyl, methoxy, ethoxy, methylthio, ethylthio, difluoromethylthio, trifluoromethylthio, C₁-C₂-haloalkyl or C₁-C₂-haloalkoxy having in each case 1 to 5 fluorine, chlorine and/or bromine atoms.
R¹⁹ particularly preferably represents fluorine, chlorine, bromine, iodine, hydroxyl, cyano, methyl, ethyl, n-propyl, isopropyl, n-butyl, iso-butyl, sec-butyl, tert-butyl, trifluoromethyl, difluoromethyl, difluorochloromethyl, trichloromethyl, methoxy, ethoxy, methylthio, ethylthio, difluoromethylthio, trifluoromethylthio, trifluoromethoxy, difluoromethoxy, difluorochloromethoxy or trichloromethoxy.
R¹⁹ very particularly preferably represents fluorine, chlorine, bromine, iodine, methyl, trifluoromethyl, difluoromethyl or trichloromethyl.
R²⁰ preferably represents hydrogen, fluorine, chlorine, bromine, iodine, cyano, C₁-C₄-alkyl, methoxy, ethoxy, methylthio, ethylthio, C₁-C₂-haloalkyl or C₁-C₂-haloalkoxy having in each case 1 to 5 fluorine, chlorine and/or bromine atoms, C₁-C₂-alkylsulphinyl or C₁-C₂-alkylsulphonyl.
R²⁰ particularly preferably represents hydrogen, fluorine, chlorine, bromine, iodine, cyano, n-propyl, isopropyl, n-butyl, isobutyl, sec-butyl, tert-butyl, trifluoromethyl, difluoromethyl, difluorochloromethyl, trichloromethyl, methoxy, ethoxy, methylthio, ethylthio, trifluoromethoxy, difluoromethoxy, difluorochloromethoxy, trichloromethoxy, methylsulphinyl or methylsulphonyl. R²⁰ very particularly preferably represents hydrogen, fluorine, chlorine, bromine, iodine, n-propyl, isopropyl, n-butyl, isobutyl, sec-butyl, tert-butyl, trifluoromethyl, difluoromethyl, trichloromethyl, methylsulphinyl or methylsulphonyl.

R²⁰ especially preferably represents hydrogen.
R²¹ preferably represents methyl, ethyl or C₁-C₂-haloalkyl having 1 to 5 fluorine, chlorine and/or bromine atoms.
R²¹ particularly preferably represents methyl, ethyl, trifluoroethyl, difluoromethyl, difluorochloromethyl or trichloromethyl.
R²² preferably represents methyl or ethyl.
R²² particularly preferably represents methyl.
Q¹ preferably represents S (sulphur), SO₂ or CH₂.
Q¹ particularly preferably represents S (sulphur) or CH₂.
Q¹ very particularly preferably represents S (sulphur).
p preferably represents 0 or 1.
p particularly preferably represents 0.
R²³ preferably represents methyl, ethyl or C₁-C₂-haloalkyl having 1 to 5 fluorine, chlorine and/or bromine atoms.
R²³ particularly preferably represents methyl, ethyl, trifluoromethyl, difluoromethyl, difluorochloromethyl or trichloromethyl.
R²³ very particularly preferably represents methyl, trifluoromethyl, difluoromethyl or trichloromethyl.
R²⁴ preferably represents methyl, ethyl or C₁-C₂-haloalkyl having 1 to 5 fluorine, chlorine and/or bromine atoms.
R²⁴ particularly preferably represents methyl, ethyl, trifluoromethyl, difluoromethyl, difluorochloromethyl or trichloromethyl.
R²⁴ very particularly preferably represents methyl, trifluoromethyl, difluoromethyl or trichloromethyl.
R²⁵ and R²⁶ independently of one another preferably represent hydrogen, fluorine, chlorine, bromine, amino, methyl, ethyl or C₁-C₂-haloalkyl having 1 to 5 fluorine, chlorine and/or bromine atoms.
R²⁵ and R²⁶ independently of one another particularly preferably represent hydrogen, fluorine, chlorine, bromine, methyl, ethyl, trifluoromethyl, difluoromethyl, difluorochloromethyl or trichloromethyl.
R²⁵ and R²⁶ independently of one another ver particularly preferably represent hydrogen, fluorine, chlorine, bromine, methyl, trifluoromethyl, difluoromethyl or trichloromethyl.
R²⁵ and R²⁶ especially preferably each represent hydrogen.
R²⁷ preferably represents hydrogen, methyl, ethyl or C₁-C₂-haloalkyl having 1 to 5 fluorine, chlorine and/or bromine atoms.
R²⁷ particularly preferably represents hydrogen, methyl, ethyl, trifluoromethyl, difluoromethyl, difluorochloromethyl or trichloromethyl.
R²⁷ very particularly preferably represents hydrogen, methyl, trifluoromethyl, difluoromethyl or trichloromethyl.
R²⁷ especially preferably represents methyl.
R²⁸ and R²⁹ independently of one another preferably represent hydrogen, fluorine, chlorine, bromine, amino, nitro, methyl, ethyl or C₁-C₂-haloalkyl having 1 to 5 fluorine, chlorine and/or bromine atoms.
R²⁸ and R²⁹ independently of one another particularly preferably represent hydrogen, fluorine, chlorine, bromine, nitro, methyl, ethyl, trifluoromethyl, difluoromethyl, difluorochloromethyl or trichloromethyl.
R²⁸ and R²⁹ independently of one another very particularly preferably represent hydrogen, fluorine, chlorine, bromine, methyl, trifluoromethyl, difluoromethyl or trichloromethyl.
R²⁸ and R²⁹ especially preferably each represent hydrogen.
R³⁰ preferably represents hydrogen, fluorine, chlorine, bromine, methyl, ethyl or C₁-C₂-haloalkyl having 1 to 5 fluorine, chlorine and/or bromine atoms,
R³⁰ particularly preferably represents hydrogen, fluorine, chlorine, bromine, methyl, ethyl, trifluoromethyl, difluoromethyl, difluorochloromethyl or trichloromethyl.
R³⁰ very particularly preferably represents hydrogen, fluorine, chlorine, bromine, methyl, trifluoromethyl, difluoromethyl or trichloromethyl.
R³⁰ especially preferably represents methyl.
R³¹ preferably represents hydrogen, fluorine, chlorine, bromine, amino, C₁-C₄-alkylamino, di(C₁-C₄-alkyl)amino, cyano, methyl, ethyl or C₁-C₂-haloalkyl having 1 to 5 fluorine, chlorine and/or bromine atoms.
R³¹ particularly preferably represents hydrogen, fluorine, chlorine, bromine, amino, methylamino, dimethylamino, cyano, methyl, ethyl, trifluoromethyl, difluoromethyl, difluorochloromethyl or trichloromethyl.
R³¹ very particularly preferably represents hydrogen, fluorine, chlorine, bromine, amino, methylamino, dimethylamino, methyl, trifluoromethyl, difluoromethyl or trichloromethyl.
R³¹ especially preferably represents amino, methylamino, dimethylamino, methyl or trifluoromethyl.
R³² preferably represents fluorine, chlorine, bromine, methyl, ethyl or C₁-C₂-haloalkyl having 1 to 5 fluorine, chlorine and/or bromine atoms.
R³² particularly preferably represents fluorine, chlorine, bromine, methyl, ethyl, trifluoromethyl, difluoromethyl, difluorochloromethyl or trichloromethyl.
R³² very particularly preferably represents fluorine, chlorine, bromine, methyl, trifluoromethyl, difluoromethyl or trichloromethyl.
R³² especially preferably represents methyl, trifluoromethyl or difluoromethyl.
R³³ preferably represents hydrogen, fluorine, chlorine, bromine, amino, C₁-C₄-alkylamino, di(C₁-C₄-alkyl)amino, cyano, methyl, ethyl or C₁-C₂-haloalkyl having 1 to 5 fluorine, chlorine and/or bromine atoms.
R³³ particularly preferably represents hydrogen, fluorine, chlorine, bromine, amino, methylamino, dimethylamino, cyano, methyl, ethyl, trifluoromethyl, difluoromethyl, difluorochloromethyl or trichloromethyl.
R³³ very particularly preferably represents hydrogen, fluorine, chlorine, bromine, amino, methylamino, dimethylamino, methyl, trifluoromethyl, difluoromethyl or trichloromethyl.
R³³ especially preferably represents amino, methylamino, dimethylamino, methyl or trifluoromethyl.
R³⁴ preferably represents fluorine, chlorine, bromine, methyl, ethyl or C₁-C₂-haloalkyl having 1 to 5 fluorine, chlorine and/or bromine atoms.
R³⁴ particularly preferably represents fluorine, chlorine, bromine, methyl, ethyl, trifluoromethyl, difluoromethyl, difluorochloromethyl or trichloromethyl.
R³⁴ very particularly preferably represents fluorine, chlorine, bromine, methyl, trifluoromethyl, difluoromethyl or trichloromethyl.
R³⁴ especially preferably represents methyl, trifluoromethyl or difluoromethyl.
R³⁵ preferably represents fluorine, chlorine, bromine, methyl, ethyl or C₁-C₂-haloalkyl having 1 to 5 fluorine, chlorine and/or bromine atoms.
R³⁵ particularly preferably represents fluorine, chlorine, bromine, methyl, ethyl, trifluoromethyl, difluoromethyl, difluorochloromethyl or trichloromethyl.
R³⁵ very particularly preferably represents fluorine, chlorine, bromine, methyl, trifluoromethyl, difluoromethyl or trichloromethyl.
R³⁶ preferably represents hydrogen, methyl or ethyl.
R³⁶ particularly preferably represents methyl.
R³⁷ preferably represents fluorine, chlorine, bromine, methyl or ethyl.
R³⁷ particularly preferably represents fluorine, chlorine or methyl.
R³⁸ preferably represents methyl, ethyl or C₁-C₂-haloalkyl having 1 to 5 fluorine, chlorine and/or bromine atoms.
R³⁸ particularly preferably represents methyl, ethyl, trifluoromethyl, difluoromethyl, difluorochloromethyl or trichloromethyl.
R³⁸ very particularly preferably represents methyl, trifluoromethyl, difluoromethyl or trichloromethyl.
R³⁸ especially preferably represents methyl or trifluoromethyl.
R³⁹ preferably represents hydrogen, fluorine, chlorine, bromine, methyl, ethyl or C₁-C₂-haloalkyl having 1 to 5 fluorine, chlorine and/or bromine atoms.
R³⁹ particularly preferably represents hydrogen, fluorine, chlorine, bromine, methyl or trifluoromethyl.
R⁴⁰ preferably represents fluorine, chlorine, bromine, iodine, hydroxyl, C₁-C₄-alkyl, methoxy, ethoxy, methylthio, ethylthio, difluoromethylthio, trifluoromethylthio, C₁-C₂-haloalkyl or C₁-C₂-haloalkoxy having in each case 1 to 5 fluorine, chlorine and/or bromine atoms.
R⁴⁰ particularly preferably represents fluorine, chlorine, bromine, iodine, methyl, ethyl, n-propyl, isopropyl, n-butyl, isobutyl, sec-butyl, tert-butyl, trifluoromethyl, difluoromethyl, difluorochloromethyl or trichloromethyl.
R⁴⁰ very particularly preferably represents fluorine, chlorine, bromine, iodine, methyl, trifluoromethyl, difluoromethyl or trichloromethyl.
R⁴¹ preferably represents methyl, ethyl, n-propyl or isopropyl.
R⁴¹ particularly preferably represents methyl or ethyl.

Preference is given to those compounds of the formula (I) in which all radicals each have the preferred meanings mentioned above.

Particular preference is given to those compounds of the formula (I) in which all radicals each have the particularly preferred meanings mentioned above.

Preference is furthermore given to compounds of the formula (I-b)

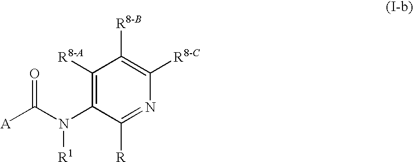

in which
R, R¹ and A are as defined above,
R^{8-A}, R^{8-B} and R^{8-C} each independently of one another have the meanings of R⁸.

Particular preference is given to compounds of the formula (I-b) in which the radicals R^{8-A}, R^{8-B} and R^{8-C} all represent hydrogen.

Furthermore, particular preference is given to compounds of the formula (I-b) in which the radicals R^{8-B} and R^{8-C} both represent hydrogen and the radical R^{8-A} represents fluorine or chlorine.

Furthermore, particular preference is given to compounds of the formula (I-b) in which the radicals R^{8-A} and R^{8-B} both represent hydrogen and the radical R^{8-C} represents methyl.

Furthermore, preference is given to the compounds of the formula (I-c)

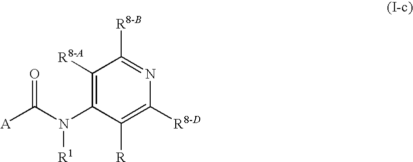

in which
R, R¹ and A are as defined above,
R^{8-A}, R^{8-B} and R^{8-D} each independently of one another have the meanings of R⁸.

Particular preference is given to compounds of the formula (I-c) in which the radicals R^{8-A}, R^{8-B} and R^{8-D} all represent hydrogen.

Furthermore, particular preference is given to compounds of the formula (I-c) in which the radicals R^{8-B} and R^{8-D} both represent hydrogen and the radical R^{8-A} represents fluorine or chlorine.

Furthermore, particular preference is given to compounds of the formula (I-c), in which the radicals R^{8-A} and R^{8-B} both represent hydrogen and the radical R^{8-D} represents methyl.

Furthermore, preference is given to compounds of the formula (I-d)

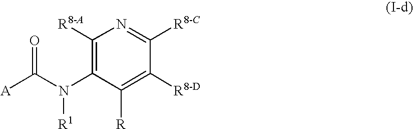

in which
R, R¹ and A are as defined above,
R^{8-A}, R^{8-C} and R^{8-D} each independently of one another have the meanings of R⁸.

Particular preference is given to compounds of the formula (I-d) in which the radicals R^{8-A}, R^{8-C} and R^{8-D} all represent hydrogen.

Furthermore, particular preference is given to compounds of the formula (I-d) in which the radicals R^{8-A} and R^{8-C} both represent hydrogen and the radical R^{8-D} represents fluorine, chlorine or methyl.

Furthermore, preference is given to the compounds of the formula (I-e)

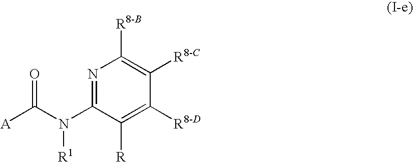

in which
R, R¹ and A are as defined above,
R^{8-B}, R^{8-C} and R^{8-D} each independently of one another have the meanings of R⁸.

Particular preference is given to compounds of the formula (I-e) in which the radicals R^{8-B}, R^{8-C} and R^{8-D} all represent hydrogen.

Furthermore, particular preference is given to compounds of the formula (I-e) in which the radicals R^{8-B} and R^{8-C} both represent hydrogen and the radical R^{8-D} represents fluorine, chlorine or methyl.

Furthermore, preference is given to compounds of the formula (I-f)

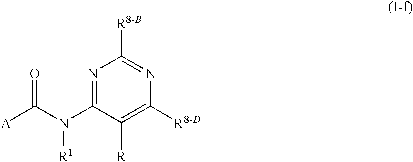

in which
R, R¹ and A are as defined above,
R^{8-B} and R^{8-D} each independently of one another have the meanings of R⁸.

Particular preference is given to compounds of the formula (I-f) in which the radicals R^{8-B} and R^{8-D} both represent hydrogen.

Moreover, particular preference is given to compounds of the formula (I-f) in which the radical R^{8-B} represents hydrogen and the radical R^{8-D} represents methyl or trifluoromethyl.

Furthermore, preference is given to compounds of the formula (I-g)

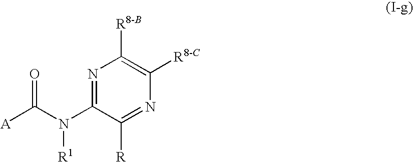

in which
R, R¹ and A are as defined above,
R^{8-B} and R^{8-C} each independently of one another have the meanings of R⁸.

Particular preference is given to compounds of the formula (I-g) in which the radicals R^{8-B} and R^{8-C} both represent hydrogen.

Furthermore, particular preference is given to compounds of the formula (I-g) in which the radical R^{8-B} represents hydrogen and the radical R^{8-C} represents methyl or trifluoromethyl.

Furthermore, preference is given to compounds of the formula (I-h)

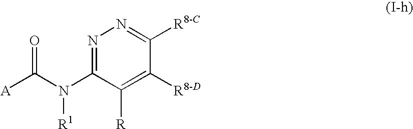

in which
R, R¹ and A are as defined above,
R^{8-C} and R^{8-D} each independently of one another have the meanings of R⁸.

Particular preference is given to compounds of the formula (I-h) in which the radicals R^{8-C} and R^{8-D} both represent hydrogen.

Furthermore, particular preference is given to compounds of the formula (I-h) in which the radical R^{8-C} represents hydrogen and the radical R^{8-D} represents methyl or trifluoromethyl.

Furthermore, preference is given to compounds of the formula (I-j)

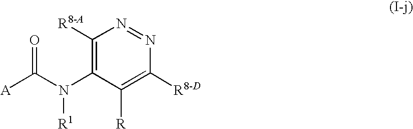

in which
R, R¹ and A are as defined above,
R^{8-A} and R^{8-D} each independently of one another have the meanings of R⁸.

Particular preference is given to compounds of the formula (I-j) in which the radicals R^{8-A} and R^{8-D} both represent hydrogen.

Furthermore, particular preference is given to compounds of the formula (I-j) in which the radical R^{8-A} represents hydrogen and the radical R^{8-D} represents methyl or trifluoromethyl.

Preference is likewise given to compounds of the formulae (I), (I-b), (I-c), (I-d), (I-e), (I-f), (I-g), (I-h), (I-j) in which R¹ represents hydrogen.

However, the general or preferred radical definitions or illustrations given above can also be combined with one another as desired, i.e. including combinations between the respective ranges and preferred ranges. They apply to the end products and, correspondingly, to the precursors and intermediates.

Using, for example, 2-(trifluoromethyl)benzoyl chloride and 5-(4-chlorophenyl)-4-pyrimidinylamine as starting materials and a base, the course of the process (a) according to the invention can be illustrated by the reaction equation below:

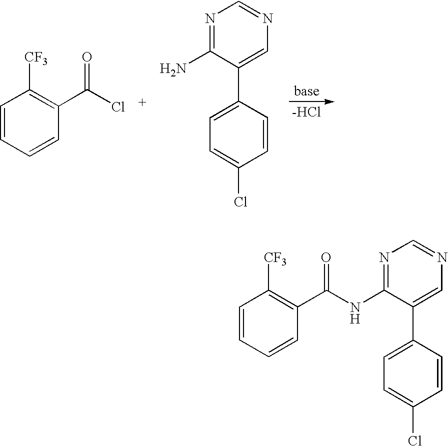

The formula (II) provides a general definition of the carbonyl halides required as starting materials for carrying out the process (a) according to the invention. In this formula (II), A preferably, particularly preferably, very particularly preferably and especially preferably has those meanings which have already been mentioned in connection with the description of the compounds of the formula (I) according to the invention as being preferred, particularly preferred, etc., for this radical. X⁵ preferably represents fluorine, chlorine or hydroxyl, particularly preferably chlorine or hydroxyl.

The carbonyl halides of the formula (II) are known and/or can be prepared by known processes (cf., for example, EP-A 0 545 099, JP-A 01-290662 and U.S. Pat. No. 5,093,347).

The formula (III) provides a general definition of the aniline derivatives furthermore required as starting materials for carrying out the process (a) according to the invention. In this formula (III), R, R¹, X¹, X², X³ and X⁴ preferably, particularly preferably, very particularly preferably and especially preferably have those meanings which have already been mentioned in connection with the description of the compounds of the formula (I) according to the invention as being preferred, particularly preferred, etc., for these radicals.

Some of the aniline derivatives of the formula (III) are known and/or they can be prepared by known processes (cf., for example, J. Org. Chem. 1972, 21, 3216-3220).

Novel, and thus also part of the subject-matter of the present application, are aniline derivatives of the formula (III-a)

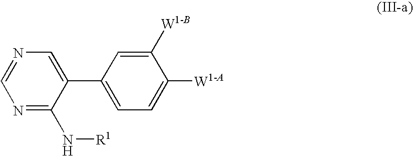

in which
R¹ is as defined above
and
W^{1-A} and W^{1-B} either both represent chlorine
or
W^{1-A} represents trifluoromethyl or trifluoromethoxy and W^{1-B} represents hydrogen.

Aniline derivatives of the formula (III-a) are obtained when

(c) phenylacetonitriles of the formula (V)

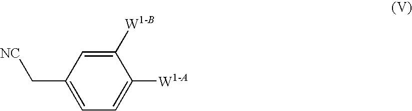

in which W^{1-A} and W^{1-B} are as defined above
are reacted with tris(formamino)methane and formamide
and the resulting aniline derivatives of the formula (III-b)

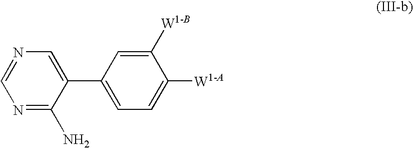

in which W^{1-A} and W^{1-B} are as defined above are, if appropriate,
reacted with halides of the formula (IV)

R^{1-B}-Hal (IV)
in which R^{1-B} and Hal are as defined above
in the presence of a base and in the presence of a diluent [for this step, the reaction conditions of process (b) apply in a corresponding manner].

The phenylacetonitriles of the formula (V) required as starting materials for carrying out the process (c) according to the invention are known chemicals for syntheses.

Using N-[5-(4-chlorophenyl)pyrimidin-4-yl]-2-(trifluoromethyl)benzamide and 2-(trifluoromethyl)benzoyl chloride as starting materials, the course of the process (b) according to the invention can be illustrated by the formula scheme below:

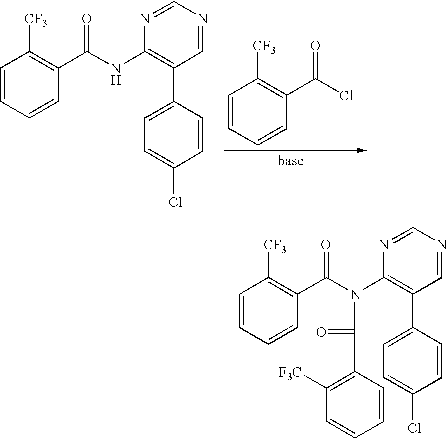

The formula (I-a) provides a general definition of the carboxamides required as starting materials for carrying out the process (b) according to the invention. In this formula (I-a), R, X¹, X², X³, X⁴ and A preferably, particularly preferably, very particularly preferably and especially preferably have those meanings which have already been mentioned in connection with the description of the compounds of the formula (I) according to the invention as being preferred, particularly preferred, etc., for these radicals.

The compounds of the formula (I-a) are compounds according to the invention and can be prepared by process (a).

The formula (IV) provides a general definition of the halides furthermore required as starting materials for carrying out the process (b) according to the invention. In this formula (IV), R^{1-B} preferably, particularly preferably, very particularly preferably and especially preferably has those meanings which have already been mentioned above for the radical R¹ as being preferred, particularly preferred, etc., where R^{1-B} never represents hydrogen. Hal represents chlorine, bromine or iodine.

Halides of the formula (IV) are known.

Suitable diluents for carrying out the process (a) according to the invention are all inert organic solvents. These preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; ketones, such as acetone, butanone, methyl isobutyl ketone or cyclohexanone; nitriles, such acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; mixtures thereof with water or pure water.

The process (a) according to the invention is, if appropriate, carried out in the presence of a suitable acid acceptor. Suitable acid acceptors are all customary inorganic or organic bases. These preferably include alkaline earth metal or alkali metal hydrides, hydroxides, amides, alkoxides, acetates, carbonates or bicarbonates, such as, for example, sodium hydride, sodium amide, lithium diisopropylamide, sodium methoxide, sodium ethoxide, potassium tert-butoxide, sodium hydroxide, potassium hydroxide, sodium acetate, sodium carbonate, potassium carbonate, potassium bicarbonate, sodium bicarbonate or ammonium carbonate, and also tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

If appropriate (if X⁵ represents hydroxyl), the process (a) according to the invention is carried out in the presence of a suitable coupling agent. Suitable coupling agents are all customary carbonyl activators. These preferably include N-[3-(dimethylamino)propyl]-N′-ethylcarbodiimide hydrochloride, N,N′-di-sec-butylcarbodiimide, N,N′-dicyclohexylcarbodiimide, N,N′-diisopropylcarbodiimide, 1-(3-(dimethylamino)propyl)-3-ethylcarbodiimide methiodide, 2-bromo-3-ethyl-4-methylthiazolium tetrafluoroborate, N,N-bis[2-oxo-3-oxazolidinyl]phosphorodiamidic chloride, chlorotripyrrolidino-phosphonium hexafluorophosphate, bromotripyrrolidinophosphonium hexafluorophosphate, O-(1H-benzotriazol-1-yloxy)tris(dimethylamino)phosphonium hexafluorophosphate, N,N,N′,N′-bis(tetramethylene)chlorouronium tetrafluoroborate, O-(1H-benzotriazol-1-yl)-N,N,N′,N′-tetramethyluronium hexafluorophosphate, O-(1H-benzotriazol-1-yl)-N,N,N′,N′-bis(tetramethylene)uronium hexafluorophosphate, O-(1H-benzotriazol-1-yl)-N,N,N′,N′-bis(tetramethylene)uronium tetrafluoroborate, O-(7-azabenzotriazol-1-yl)-N,N,N,N-tetramethyluronium hexafluorophosphate and 1-hydroxybenzotriazole. These reagents can be employed separately, but also in combination.

When carrying out the process (a) according to the invention, the reaction temperatures may be varied within a relatively wide range. In general, the process is carried out at temperatures of from 0° C. to 150° C., preferably at temperatures of from 20° C. to 110° C.

For carrying out the process (a) according to the invention for preparing the compounds of the formula (I), in general from 0.2 to 5 mol, preferably from 0.5 to 2 mol, of aniline derivative of the formula (III) are employed per mole of the carbonyl halide. Work-up is carried out by customary methods.

Suitable diluents for carrying out the process (b) according to the invention are all inert organic solvents. These preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl tert-butyl ether, methyl tert-amyl ether, dioxane, tetrahydrofuran, 1,2-dimethoxyethane, 1,2-diethoxyethane or anisole, or amides, such as N,N-dimethylformamide, N,N-dimethylacetamide, N-methylformanilide, N-methylpyrrolidone or hexamethylphosphoric triamide.

The process (b) according to the invention is carried out in the presence of a base. Suitable bases are all customary inorganic or organic bases. These preferably include alkaline earth metal or alkali metal hydrides, hydroxides, amides, alkoxides, acetates, carbonates or bicarbonates, such as, for example, sodium hydride, sodium amide, sodium methoxide, sodium ethoxide, potassium tert-butoxide, sodium hydroxide, potassium hydroxide, ammonium hydroxide, sodium acetate, potassium acetate, calcium acetate, ammonium acetate, sodium carbonate, potassium carbonate, potassium bicarbonate, sodium bicarbonate or caesium carbonate, and also tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

When carrying out the process (b) according to the invention, the reaction temperatures can be varied within a relatively wide range. In general, the process is carried out at temperatures of from 0° C. to 150° C., preferably at temperatures of from 20° C. to 110° C.

For carrying out the process (b) according to the invention for preparing the compounds of the formula (I), in general from 0.2 to 5 mol, preferably from 0.5 to 2 mol, of a halide of the formula (IV) are employed per mole of the carboxamide of the formula (I-a).

Suitable diluents for carrying out the process (c) according to the invention are all inert organic solvents. These preferably include aliphatic, alicyclic or aromatic hydrocarbons, such as, for example, petroleum ether, hexane, heptane, cyclohexane, methylcyclohexane, benzene, toluene, xylene or decalin; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as acetonitrile, propionitrile, n- or i-butyronitrile or benzonitrile; amides, such has N,N-dimethylformamide, N,N-dimethylacetamide, N-methylformanilide, N-methylpyrrolidone or hexamethylphosphoric triamide; esters, such as methyl acetate or ethyl acetate; sulphoxides, such as dimethyl sulphoxide; sulphones, such as sulpholane; alcohols, such as methanol, ethanol, n- or i-propanol, n-, i-, sec- or tert-butanol, ethanediol, propane-1,2-diol, ethoxyethanol, methoxyethanol, diethylene glycol monomethyl ether, diethylene glycol monoethyl ether, mixtures thereof with water or pure water.

When carrying out the process (c) according to the invention, the reaction temperatures can be varied within a relatively wide range. In general, the process is carried out at temperatures of from 100° C. to 250° C., preferably at temperatures of from 140° C. to 200° C.

The process (c) according to the invention is, if appropriate, carried out in the presence of a suitable acid. Suitable acids are all customary inorganic or organic acids. Preference is given to using hydrochloric acid, sulphuric acid, hydrofluoric acid, phosphoric acid, p-toluenesulphonic acid, methanesulphonic acid, trifluoroacetic acid or trichloroacetic acid. Particular preference is given to using hydrochloric acid, sulphuric acid, p-toluenesulphonic acid or trifluoroacetic acid, very particularly preferably p-toluenesulphonic acid. If appropriate, the acid used can be employed as a mixture with water.

For carrying out the process (c) according to the invention for preparing the compounds of the formula (III-a), in general from 1 to 10 mol, preferably from 1 to 3 mol, of tris(formamino)methane and from 1 to 100 mol, preferably from 3 to 10 mol, of formamide are employed per mole of the phenylacetonitrile of the formula (V). Work-up is carried out by customary methods.

The processes (a), (b) and (c) according to the invention are generally carried out under atmospheric pressure. However, it is also possible to operate under elevated or reduced pressure—in general between 0.1 bar and 10 bar.

The compounds according to the invention exhibit a potent microbicidal activity and can be employed in crop protection and in the protection of materials for controlling undesirable microorganisms such as fungi and bacteria.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Examples which may be mentioned, but not by limitation, of some pathogens of fungal and bacterial diseases which come under the abovementioned general terms are:

diseases caused by powdery mildew pathogens, such as, for example
Blumeria species such as, for example, Blumeria graminis;
Podosphaera species such as, for example, Podosphaera leucotricha;
Sphaerotheca species such as, for example, Sphaerotheca fuliginea;
Uncinula species such as, for example, Uncinula necator;
diseases caused by rust pathogens such as, for example,
Gymnosporangium species such as, for example, Gymnosporangium sabinae
Hemileia species such as, for example, Hemileia vastatrix;
Phakopsora species such as, for example, Phakopsora pachyrhizi and Phakopsora meibomiae;
Puccinia species such as, for example, Puccinia recondita or Puccinia graminis;
Uromyces species such as, for example, Uromyces appendiculatus;
diseases caused by pathogens from the Oomycetes group such as, for example,
Bremia species such as, for example, Bremia lactucae;
Peronospora species such as, for example, Peronospora pisi or P. brassicae;
Phytophthora species such as, for example, Phytophthora infestans;
Plasmopara species such as, for example, Plasmopara viticola;
Pseudoperonospora species such as, for example, Pseudoperonospora humuli or Pseudoperonospora cubensis;
Pythium species such as, for example, Pythium ultimum;
leaf spot diseases and leaf wilts caused by, for example,
Alternaria species such as, for example, Alternaria solani;
Cercospora species such as, for example, Cercospora beticola;
Cladosporium species such as, for example, Cladosporium cucumerinum;
Cochliobolus species such as, for example, Cochliobolus sativus (conidial form: Drechslera, syn: Helminthosporium);
Colletotrichum species such as, for example, Colletotrichum lindemuthanium;
Cycloconium species such as, for example, Cycloconium oleaginum;
Diaporthe species such as, for example, Diaporthe citri;
Elsinoe species such as, for example, Elsinoe fawcettii;
Gloeosporium species such as, for example, Gloeosporium laeticolor;
Glomerella species such as, for example, Glomerella cingulata;
Guignardia species such as, for example, Guignardia bidwelli;
Leptosphaeria species such as, for example, Leptosphaeria maculans;
Magnaporthe species such as, for example, Magnaporthe grisea;
Mycosphaerella species such as, for example, Mycosphaerella graminicola and Mycosphaerella fijiensis;
Phaeosphaeria species such as, for example, Phaeosphaeria nodorum;
Pyrenophora species such as, for example, Pyrenophora teres;
Ramularia species such as, for example, Ramularia collo-cygni;
Rhynchosporium species such as, for example, Rhynchosporium secalis;
Septoria species such as, for example, Septoria apii;
Typhula species such as, for example, Typhula incamata;
Venturia species such as, for example, Venturia inaequalis;
root and stem diseases caused by, for example,
Corticium species such as, for example, Corticium graminearum;
Fusarium species such as, for example, Fusarium oxysporum;
Gaeumannomyces species such as, for example, Gaeumannomyces graminis;
Rhizoctonia species such as, for example, Rhizoctonia solani;
Tapesia species such as, for example, Tapesia acuformis or Tapesia yallundae;
Thielaviopsis species such as, for example, Thielaviopsis basicola;
ear and panicle diseases (including maize cobs), caused by, for example,
Alternaria species such as, for example, Alternaria spp.;
Aspergillus species such as, for example, Aspergillus flavus;
Cladosporium species such as, for example, Cladosporium cladosporioides;
Claviceps species such as, for example, Claviceps purpurea;
Fusarium species such as, for example, Fusarium culmorum;
Gibberella species such as, for example, Gibberella zeae;
Monographella species such as, for example, Monographella nivalis;
diseases caused by smuts such as, for example,
Sphacelotheca species such as, for example, Sphacelotheca reiliana;
Tilletia species such as, for example, Tilletia caries;
Urocystis species such as, for example, Urocystis occulta;
Ustilago species such as, for example, Ustilago nuda;
fruit rots caused by, for example,
Aspergillus species such as, for example, Aspergillus flavus;
Botrytis species such as, for example, Botrytis cinerea;
Penicillium species such as, for example, Penicillium expansum and Penicillium purpurogenum;
Sclerotinia species such as, for example, Sclerotinia sclerotiorum;
Verticilium species such as, for example, Verticilium alboatrum;
seed- and soil-borne rot and wilts, and seedling diseases, caused by, for example,
Fusarium species such as, for example, Fusarium culmorum;
Phytophthora species such as, for example, Phytophthora cactorum;
Pythium species such as, for example, Pythium ultimum;
Rhizoctonia species such as, for example, Rhizoctonia solani;
Sclerotium species such as, for example, Sclerotium rolfsii;
cancers, galls and witches' broom disease, caused by, for example,
Nectria species such as, for example, Nectria galligena;
Wilts caused by, for example,
Monilinia species such as, for example, Monilinia laxa;
deformations of leaves, flowers and fruits, caused by, for example,
Taphrina species such as, for example, Taphrina deformans;
degenerative diseases of woody species, caused by, for example,
Esca species such as, for example, Phaeamoniella clamydospora and Phaeoacremonium alephilum and Fomitiporia mediterranea;
diseases of flowers and seeds, caused by, for example,
Botrytis species such as, for example, Botrytis cinerea;
diseases of the plant tubers, caused by, for example,
Rhizoctonia species such as, for example, Rhizoctonia solani;
Helminthosporium species such as, for example, Helminthosporium solani;
diseases caused by bacterial pathogens such as, for example,
Xanthomonas species such as, for example, Xanthomonas campestris pv. oryzae;
Pseudomonas species such as, for example, Pseudomonas syringae pv. lachrymans;
Erwinia species such as, for example, Erwinia amylovora.

The following diseases of soybeans can preferably be controlled:

Fungal diseases on leaves, stems, pods and seeds caused by, for example,
alternaria leaf spot (Alternaria spec. atrans tenuissima), anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi), scab (Sphaceloma glycines), stemphylium leaf blight (Stemphylium botryosum), target spot (Corynespora cassiicola);
fungal diseases on roots and the stem base caused by, for example,
black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulars, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

The active compounds according to the invention also have a potent strengthening effect in plants. They are therefore suitable for mobilizing the plants' defences against attack by undesired microorganisms.

Plant-strengthening (resistance-inducing) substances are understood as meaning, in the present context, those substances which are capable of stimulating the defence system of plants in such a way that, when subsequently inoculated with undesired microorganisms, the treated plants display a substantial degree of resistance to these microorganisms.

In the present case, undesired microorganisms are understood as meaning phytopathogenic fungi, bacteria and viruses. Thus, the substances according to the invention can be employed for protecting plants against attack by the abovementioned pathogens within a certain period of time after the treatment. The period of time within which their protection is effected is generally extended from 1 to 28 days, preferably 1 to 14 days, after the plants have been treated with the active compounds.

The fact that the active compounds, at the concentrations required for the controlling of plant diseases, are well tolerated by plants permits the treatment of aerial plant parts, of vegetative propagation material and seed, and of the soil.

In this context, the active compounds according to the invention can be employed particularly successfully for controlling cereal diseases such as, for example, against Puccinia species and diseases in viticulture, fruit production and vegetable production such as, for example against Botrytis, Venturia or Alternaria species.

The active compounds according to the invention are also suitable for increasing the yield. Moreover, they display a low degree of toxicity and are well tolerated by plants.

If appropriate, the active compounds according to the invention can also be used in certain concentrations and application rates as herbicides, for influencing plant growth and for controlling animal pests. If appropriate, they can also be employed as intermediates and precursors for the synthesis of further active compounds.

All plants and plant parts can be treated in accordance with the invention. Plants are understood as meaning, in the present context, all plants and plant populations, such as desired and undesired wild plants or crop plants (including naturally occurring crop plants). Crop plants may be plants which can be obtained by conventional breeding and optimization methods or else by biotechnological and genetic engineering methods or by combinations of these methods, including the transgenic plants and including the plant varieties capable or not capable of being protected by Plant Breeders' rights. Plant parts are understood as meaning all aerial and subterranean parts and organs of the plants, such as shoot, leaf, flower and root, examples which may be mentioned being leaves, needles, stalks, stems, flowers, fruiting bodies, fruits and seeds, and also roots, tubers and rhizomes. The plant parts also include harvested material and vegetative and generative propagation material, for example cuttings, tubers, rhizomes, slips and seeds.

The treatment according to the invention with the active compounds, of the plants and plant parts, is carried out directly or by acting on their environment, habitat, or store by the customary treatment methods, for example by immersion, spraying, vaporizing, fogging, broadcasting, painting on and, in the case of propagation material, in particular in the case of seeds, furthermore by coating with one or more coats.

In the protection of materials, the compounds according to the invention can be employed for protecting industrial materials against attack and destruction by undesired microorganisms.

In the present context, industrial materials are understood as meaning non live materials which have been made for use in technology. For example, industrial materials which are to be protected by active compounds according to the invention from microbial modification or destruction can be glues, sizes, paper and board, textiles, leather, timber, paints and plastic articles, cooling lubricants and other materials which are capable of being attacked or destroyed by microorganisms. Parts of production plants, for example cooling-water circuits, which can be adversely affected by the multiplication of microorganisms may also be mentioned within the materials to be protected. Industrial materials which may be mentioned with preference for the purposes of the present invention are glues, sizes, paper and board, leather, timber, paints, cooling lubricants and heat-transfer fluids, especially preferably wood.

Microorganisms which are capable of bringing about a degradation or modification of the industrial materials and which may be mentioned are, for example, bacteria, fungi, yeasts, algae and slime organisms. The active compounds according to the invention are preferably active against fungi, in particular moulds, wood-discolouring and wood-destroying fungi (Basidiomycetes) and against slime organisms and algae.

Examples which may be mentioned are microorganisms of the following genera:

Alternaria such as Alternaria tenuis,
Aspergillus such as Aspergillus niger,
Chaetomium such as Chaetomium globosum,
Coniophora such as Coniophora puetana,
Lentinus such as Lentinus tigrinus,
Penicillium such as Penicillium glaucum,
Polyporus such as Polyporus versicolor,
Aureobasidium such as Aureobasidium pullulans,
Sclerophoma such as Sclerophoma pityophila,
Trichoderma such as Trichoderma viride,
Escherichia such as Escherichia coli,
Pseudomonas such as Pseudomonas aeruginosa,
Staphylococcus such as Staphylococcus aureus.

Depending on their respective physical and/or chemical properties, the active compounds can be converted to the customary formulations, such as solutions, emulsions, suspensions, powders, foams, pastes, granules, aerosols, very fine capsules in polymeric substances and in coating compositions for seed, and also ULV cold- and warm-fogging formulations.

These formulations are produced in a known manner, for example by mixing the active compounds with extenders, that is, liquid solvents, pressurized liquefied gases and/or solid carriers, optionally with the use of surface-active agents, that is emulsifers and/or dispersants, and/or foam formers. If the extender used is water, it is also possible to employ for example organic solvents as cosolvents. Suitable liquid solvents are essentially: aromatics, such as xylene, toluene or alkylnaphthalenes, chlorinated aromatics or chlorinated aliphatic hydrocarbons, such as chlorobenzenes, chloroethylenes or methylene chloride, aliphatic hydrocarbons, such as cyclohexane or paraffins, for example mineral oil fractions, alcohols, such as butanol or glycol as well as their ethers and esters, ketones, such as acetone, methyl ethyl ketone, methyl isobutyl ketone or cyclohexanone, strongly polar solvents, such as dimethylformamide and dimethyl sulphoxide, and also water. Liquefied gaseous extenders or carriers are those liquids which are gaseous at ambient temperature and at atmospheric pressure, for example aerosol propellants such as halogenated hydrocarbons and also butane, propane, nitrogen and carbon dioxide. As solid carriers there are suitable: for example ground natural minerals, such as kaolins, clays, talc, chalk quartz, attapulgite, montmorillonite or diatomaceous earth, and ground synthetic minerals, such as finely divided silica, alumina and silicates. As solid carriers for granules there are suitable: for example crushed and fractionated natural rocks such as calcite, pumice, marble, sepiolite and dolomite, and also synthetic granules of inorganic and organic meals, and granules of organic material such as sawdust, coconut shells, maize cobs and tobacco stalks. As emulsifiers and/or foam formers there are suitable: for example non-ionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and protein hydrolysates. As dispersants there are suitable: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, as well as natural phospholipids, such as cephalins and lecithins, and synthetic phospholipids, can be used in the formulations. Other possible additives are mineral and vegetable oils.

It is possible to use colorants such as inorganic pigments, for example iron oxide, titanium oxide Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations in general contain between 0.1 and 95% by weight of active compound, preferably between 0.5 and 90%.

The active compounds according to the invention, as such or in their formulations, can also be used as a mixture with known fungicides, bactericides, acaricides, nematicides, or insecticides, for example, to improve the activity spectrum or prevent the development of resistance. In many instances, synergistic effects are obtained, i.e. the activity of the mixture exceeds the activity of the individual components.

Examples of co-components in mixtures are the following compounds:

### Fungicides:

1) Nucleic acid synthesis inhibitors: for example benalaxyl, benalaxyl-M, bupirimate, clozylacon, dimethirimol, ethirimol, furalaxyl, hymexazol, mefenoxam, metalaxyl, metalaxyl-M. ofurace, oxadixyl, oxolinic acid;
2) mitosis and cell division inhibitors: for example benomyl, carbendazim, diethofencarb, ethaboxam, fuberidazole, pencycuron, thiabendazole, thiophanate-methyl, zoxamide;
3) respiration inhibitors (inhibitors of the respiratory chain):
3.1) inhibitors which act on complex I of the respiratory chain: for example diflumetorim;
3.2) inhibitors which act on complex II of the respiratory chain: for example boscalid/nicobifen, carboxin, fenfuram, flutolanil, furametpyr, furmecyclox, mepronil, oxycarboxin, penthiopyrad, thifluzamide;
3.3) inhibitors which act on complex III of the respiratory chain: for example amisulbrom, azoxystrobin, cyazofamid, dimoxystrobin, enestrobin, famoxadone, fenamidone, fluoxastrobin, kresoxim-methyl, metominostrobin, orysastrobin, picoxystrobin, pyraclostrobin, trifloxystrobin;
4) decouplers: for example dinocap, fluazinam, meptyldinocap;
5) ATP production inhibitors: for example fentin acetate, fentin chloride, fentin hydroxide, silthiofam;
6) amino acid and protein biosynthesis inhibitors: for example andoprim, blasticidin-S, cyprodinil, kasugamycin, kasugamycin hydrochloride hydrate, mepanipyrim, pyrimethanil;
7) signal transduction inhibitors: for example fenpiclonil, fludioxonil, quinoxyfen;
8) lipid and membrane synthesis inhibitors: for example biphenyl, chlozolinate, edifenphos, iodocarb, iprobenfos, iprodione, isoprothiolane, procymidone, propamocarb, propamocarb hydrochloride, pyrazophos, tolclofos-methyl, vinclozolin;
9) ergosterol biosynthesis inhibitors: for example aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulfate, imibenconazole, ipconazole, metconazole, myclobutanil, naftifine, nuarimol, oxpoconazole, paclobutrazol, pefurazoate, penconazole, prochloraz, propiconazole, prothioconazole, pyributicarb, pyrifenox, simeconazole, spiroxamine, tebuconazole, terbinafine, tetraconazole, triadimefon, triadimenol, tridemorph, triflumizole, triforine, triticonazole, uniconazole, viniconazole, voriconazole;
10) cell wall synthesis inhibitors: for example benthiavalicarb, dimethomorph, flumorph, iprovalicarb, polyoxins, polyoxorim, validamycin A;
11) melanin biosynthesis inhibitors: for example carpropamid, diclocymet, fenoxanil, phthalide, pyroquilon, tricyclazole;
12) resistance inductors: for example acibenzolar-S-methyl, probenazole, tiadinil;
13) compounds with multi-site activity: for example Bordeaux mixture, captafol, captan, chlorothalonil, copper naphthenate, copper oxide, copper oxychloride, copper preparations such as, for example, copper hydroxide, copper sulphate, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, oxine-copper, propineb, sulphur and sulphur preparations such as, for example, calcium polysulphide, thiram, tolylfluanid, zineb, ziram;
14) a compound selected from the following enumeration: N-methyl-(2E)-2-(2-{[6-(3-chloro-2-methylphenoxy)-5-fluoropyrimidin-4-yl]oxy}phenyl)-2-(methoxyimino)acetamide, N-methyl-(2E)-2-{2-[({[(1E)-1-(3-{[(E)-1-fluoro-2-phenylvinyl]oxy}phenyl)ethylidene]amino}oxy)methyl]phenyl}-2-(methoxyimino)acetamide, 1-(4-chlorophenyl)-2-(1H-1,2,4-triazol-1-yl)cycloheptanol, 1-[(4-methoxyphenoxy)methyl]-2,2-dimethylpropyl-1H-imidazole-1-carboxylate, 2-(4-chlorophenyl)-N-{2-[3-methoxy-4-(prop-2-yn-1-yloxy)phenyl]ethyl}-2-(prop-2-yn-1-yloxy)acetamide, 2,3,5,6-tetrachloro-4-(methylsulphonyl)pyridine, 2-butoxy-6-iodo-3-propyl-4H-chromen-4-one, 2-chloro-N-(1,1,3-trimethyl-2,3-dihydro-1H-inden-4-yl)nicotinamide, 2-phenylphenol and salts thereof, 3,4,5-trichloropyridine-2,6-dicarbonitrile, 3,4-dichloro-N-(2-cyanophenyl)isothiazole-5-carboxamide, 3-[5-(4-chlorophenyl)-2,3-dimethylisoxazolidin-3-yl]pyridine, 5-chloro-6-(2,4,6-trifluorophenyl)-N-[(1R)-1,2,2-trimethylpropyl][1,2,4]triazolo[1,5-a]pyrimidine-7-amine, 5-chloro-7-(4-methylpiperidin-1-yl)-6-(2,4,6-trifluorophenyl) [1,2,4]triazolo[1,5-a]pyrimidine, 5-chloro-N-[(1R)-1,2-dimethylpropyl]-6-(2,4,6-trifluorophenyl) [1,2,4]triazolo[1,5-a]pyrimidine-7-amine, 8-hydroxyquinoline sulphate, benthiazole, bethoxazin, capsimycin, carvone, quinomethionate, cufraneb, cyflufenamid, cymoxanil, dazomet, debacarb, dichlorophen, diclomezine, dicloran, difenzoquat, difenzoquat methylsulphate, diphenylamine, ferimzone, flumetover, fluopicolide, fluoroimide, flusulfamide, fosetyl-aluminum, fosetyl-calcium, fosetyl-sodium, hexachlorobenzene, irumamycin, methasulfocarb, methyl (2-chloro-5-{(1E)-N-[(6-methylpyridin-2-yl)methoxy]ethanimidoyl}benzyl)carbamate, methyl (2E)-2-{2-[({cyclopropyl[(4-methoxyphenyl)imino]methyl}thio)methyl]phenyl}-3-methoxyacrylate, methyl 1-(2,2-dimethyl-2,3-dihydro-1H-inden-1-yl)-1H-imidazole-5-carboxylate, methyl 3-(4-chlorophenyl)-3-{[N-(isopropoxycarbonyl)valyl]amino}propanoate, methyl isothiocyanate, metrafenone, mildiomycin, N-(3′,4′-dichloro-5-fluorobiphenyl-2-yl)-3-(difluoromethyl)-1-methyl-1H-pyrazole-4-carboxamide, N-(3-ethyl-3,5,5-trimethylcyclohexyl)-3-(formylamino)-2-hydroxybenzamide, N-(4-chloro-2-nitrophenyl)-N-ethyl-4-methylbenzenesulphonamide, N-[(5-bromo-3-chloropyridin-2-yl)methyl]-2,4-dichloronicotinamide, N-[1-(5-bromo-3-chloropyridin-2-yl)ethyl]-2,4-dichloronicotinamide, N-[1-(5-bromo-3-chloropyridin-2-yl)ethyl]-2-fluoro-4-iodonicotinamide, N-[2-(4-{[3-(4-chlorophenyl)prop-2-yn-1-yl]oxy}-3-methoxyphenyl)ethyl]-N²-(methylsulphonyl)valinamide, N-{(Z)-[(cyclopropylmethoxy)imino][6-(difluoromethoxy)-2,3-difluorophenyl]methyl}-2-phenylacetamide, N-{2-[3-chloro-5-(trifluoromethyl)pyridin-2-yl]ethyl}-2-(trifluoromethyl)benzamide, natamycin, nickel dimethyl dithiocarbamate, nitrothal-isopropyl, 0-{1-[(4-methoxyphenoxy)methyl]-2,2-dimethylpropyl} 1H-imidazole-1-carbothioate, octhilinone, oxamocarb, oxyfenthiin, pentachlorophenol and salts, phosphoric acid and its salts, piperalin, propamocarb fosetylate, propanosine-sodium, proquinazid, pyrrolnitrine, quintozene, tecloftalam, tecnazene, triazoxide, trichlamide, zarilamid.

### Bactericides:

Bronopol, dichlorophen, nitrapyrin, nickel dimethyl dithiocarbamate, kasugamycin, octhilinone, furan-carboxylic acid, oxytetracyclin, probenazole, streptomycin, tecloftalam, copper sulphate and other copper preparations.

### Insecticides/Acaricides/Nematicides:

### 1. Acetylcholine Esterase (AChE) Inhibitors

1.1 Carbamates (for example alanycarb, aldicarb, aldoxycarb, allyxycarb, aminocarb, azamethiphos, bendiocarb, benfuracarb, bufencarb, butacarb, butocarboxim, butoxycarboxim, carbaryl, carbofuran, carbosulfan, chloethocarb, coumaphos, cyanofenphos, cyanophos, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, triazamate, trimethacarb, XMC, xylylcarb)
1.2 Organophosphates (for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-S-methylsulphon, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion)

### 2. Sodium Channel Modulators/Voltage-Dependent Sodium Channel Blockers

2.1 Pyrethroids (for example acrinathrin, allethrin (d-cis-trans, d-trans), beta-cyfluthrin, bifenthrin, bio-allethrin, bioallethrin-S-cyclopentyl isomer, bioethanomethrin, biopermethrin, bioresmethrin, chlo-vaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, DDT, deltamethrin, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R trans-isomer), prallethrin, profluthrin, protrifenbute, pyresmethrin, resmethrin, RU 15525, silafluofen, tau-fluvalinate, tefluthrin, terallethrin, tetramethrin (1R isomer), tralomethrin, transfluthrin, ZXI 8901, pyrethrins (pyrethrum))
2.2 Oxadiazines (for example indoxacarb)

### 3. Acetylcholine Receptor Agonists/Antagonists

3.1 Chloronicotinyls/neonicotinoids (for example acetamiprid, clothianidin, dinotefuran, imidacloprid, nitenpyram, nithiazine, thiacloprid, thiamethoxam)
3.2 Nicotine, bensultap, cartap

### 4. Acetylchuoline Receptor Modulators

4.1 Spinosyns (for example spinosad)

### 5. GABA-Controlled Chloride Channel Antagonists

5.1 Cyclodiene organochlorines (for example camphechlor, chlordane, endosulfan, gamma-HCH, HCH, heptachlor, lindane, methoxychlor
5.2 Fiproles (for example acetoprole, ethiprole, fipronil, vaniliprole)

### 6. Chloride Channel Activators

6.1 Mectins (for example abamectin, avermectin, emamectin, emamectin benzoate, ivermectin, milbemectin, milbemycin)

### 7. Juvenile Hormone Mimetics

(for example diofenolan, epofenonane, fenoxycarb, hydroprene, kinoprene, methoprene, pyriproxifen, triprene)

### 8. Ecdysone Agonists/Disruptors

8.1 Diacylhydrazines (for example chromafenozide, halofenozide, methoxyfenozide, tebufenozide)

### 9. Chitin Biosynthesis Inhibitors

9.1 Benzoylureas (for example bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron)

### 9.2 Buprofezin

### 9.3 Cyromazine

### 10. Inhibitors of Oxidative Phosphorylation, ATP Disruptors

### 10.1 Diafenthiuron

10.2 Organotins (for example azocyclotin, cyhexatin, fenbutatin oxide)

### 11. Uncouplers of Oxidative Phosphorylation by Interrupting the H-Proton Gradient

11.1 Pyrroles (for example chlorfenapyr)
11.2 Dinitrophenols (for example binapacyrl, dinobuton, dinocap, DNOC)

### 12. Site-I Electron Transport Inhibitors

12.1 METIs (for example fenazaquin, fenpyroximate, pyrimidifen, pyridaben, tebufenpyrad, tolfenpyrad)

### 12.2 Hydramethylnone

### 12.3 Dicofol

### 13. Site-II Electron Transport Inhibitors

### 13.1 Rotenone

### 14. Site-III Electron Transport Inhibitors

14.1 Acequinocyl, fluacrypyrim

### 15. Microbial Disruptors of the Insect Gut Membrane

Bacillus thuringiensis strains

### 16. Fat Synthesis Inhibitors

16.1 Tetronic acids (for example spirodiclofen, spiromesifen)
16.2 Tetramic acids [for example 3-(2,5-dimethylphenyl)-8-methoxy-2-oxo-1-azaspiro[4.5]dec-3-en-4-yl ethyl carbonate (also known as: carbonic acid, 3-(2,5-dimethylphenyl)-8-methoxy-2-oxo-1-azaspiro[4.5]dec-3-en-4-yl ethyl ester, CAS Reg. No.: 382608-10-8) and carbonic acid, cis-3-(2,5-dimethylphenyl)-8-methoxy-2-oxo-1-azaspiro[4.5]dec-3-en-4-yl ethyl ester (CAS Reg.-No.: 203313-25-1)]

### 17. Carboxamides

(for example flonicamid)

### 18. Octopaminergic Agonists

(for example amitraz)

### 19. Inhibitors of Magnesium-Stimulated ATPase

(for example propargite)

### 20. Ryanodin Receptor Agonists

20.1 Benzoic acid dicarboxamides
[for example N²-[1,1-dimethyl-2-(methylsulfonyl)ethyl]-3-iodo-N′-[2-methyl-4-[1,2,2,2-tetrafluoro-1-(trifluoromethyl)ethyl]phenyl]-1,2-benzenedicarboxamide (CAS Reg. No.: 272451-65-7), flubendiamide]
20.2 Anthranilamides (for example DPX E2Y45=3-bromo-N-{4-chloro-2-methyl-6-[(methylamino)-carbonyl]phenyl}-1-(3-chloropyridin-2-yl)-1H-pyrazole-5-carboxamide)

### 21. Nereistoxin Analogues

(for example thiocyclam hydrogen oxalate, thiosultap sodium)

### 22. Biologicals, Hormones or Pheromones

(for example azadirachtin, Bacillus spec., Beauveria spec., codlemone, Metarrhizium spec., Paecilomyces spec., thuringiensin, Verticillium spec.)
23. Active Compounds with Unknown or Unspecific Mechanisms of Action
23.1 Fumigants (for example aluminium phosphide, methyl bromide, sulphuryl fluoride)
23.2 Selective antifeedants (for example cryolite, flonicamid, pymetrozine)
23.3 Mite growth inhibitors (for example clofentezine, etoxazole, hexythiazox)
23.4 Amidoflumet, benclothiaz, benzoximate, bifenazate, bromopropylate, buprofezin, quinomethionate, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyrafluprole, pyridalyl, pyriprole, sulfluramid, tetradifon, tetrasul, triarathene, verbutin, furthermore the compound 3-methyl-phenyl propylcarbamate (tsumacide Z), the compound 3-(5-chloro-3-pyridinyl)-8-(2,2,2-trifluoroethyl)-8-azabicyclo[3.2.1]octane-3-carbonitrile (CAS Reg. No. 185982-80-3) and the corresponding 3-endo isomer (CAS Reg. No. 185984-60-5) (cf. WO 96/37494, WO 98/25923), and preparations which contain insecticidally active plant extracts, nematodes, fungi or viruses.

A mixture with other known active compounds such as herbicides, or with fertilizers and growth regulators, safeners or semiochemicals is also possible.

In addition, the compounds of the formula (I) according to the invention also have very good antimycotic activity. They have a very broad antimycotic spectrum of action, in particular against dermatophytes and budding fungi, moulds and diphasic fungi (for example against Candida species such as Candida albicans, Candida glabrata) and Epidermophyton floccosum, Aspergillus species such as Aspergillus niger and Aspergillus fumigatus, Trichophyton species such as Trichophyton mentagrophytes, Microsporon species such as Microsporon canis and audouinii. The enumeration of these fungi is no restriction whatsoever of the mycotic spectrum which can be controlled and is provided by illustration only.

The active compounds can be employed as such, in the form of their formulations or the use forms prepared therefrom, such as ready-to-use solutions, suspensions, wettable powders, pastes, soluble powders, dusts and granules. They are applied in the customary manner, for example by pouring, spraying, atomizing, broadcasting, dusting, foaming, painting on and the like. It is furthermore possible to apply the active compounds by the ultra-low-volume method, or to inject the active compound preparation or the active compound itself into the soil. The seed of the plant can also be treated.

When employing the active compounds according to the invention as fungicides, the application rates can be varied within a substantial range, depending on the type of application. In the treatment of plant parts, the application rates of active compound are generally between 0.1 and 10 000 g/ha, preferably between 10 and 1000 g/ha. For the treatment of seed, the application rates of active compound are generally between 0.001 and 50 g per kilogram of seed, preferably between 0.01 and 10 g per kilogram of seed. For treating the soil, the application rates of active compound are generally between 0.1 and 10 000 g/ha, preferably between 1 and 5000 g/ha.

As already mentioned above, all plants and their parts can be treated in accordance with the invention. In a preferred embodiment, plant species and plant varieties which are found in the wild or are obtained by traditional biological breeding methods, such as hybridization or protoplast fusion, and parts of the former are treated. In a further preferred embodiment, transgenic plants and plant varieties which have been obtained by recombinant methods, if appropriate in combination with traditional methods (genetically modified organisms) and their parts are treated. The term “parts” or “parts of plants” or “plant parts” has been illustrated above.

Particularly preferably, plants of the plant cultivars which are in each case commercially available or in use are treated according to the invention. Plant cultivars are understood as meaning plants with new properties (“traits”) which have been obtained by conventional cultivation, by mutagenesis or else by recombinant DNA techniques. These may be cultivars, breeds, biotypes or genotypes.

Depending on the plant species or plant cultivars, their location and growth conditions (soils, climate, vegetation period, nutrition), the treatment according to the invention may also result in superadditive (“synergistic”) effects. Thus, for example, reduced application rates and/or extensions of the activity spectrum and/or an increase in the activity of the substances and compositions that can be used according to the invention, better plant growth, more developed root system, higher resistance of the plant variety or plant cultivar, increased growth of shoots, higher plant vitality, increased tolerance to high or low temperatures, increased tolerance to drought or to water or soil salinity, increased flowering performance, easier harvesting, accelerated maturation, higher harvest yields, larger fruit, increased plant size, greener leaf colour, earlier blossoming, better quality and/or a higher nutritional value of the harvested products, higher sugar concentration in the fruits, better storage stability and/or processability of the harvested products which exceed the effects which were actually to be expected are possible.

The preferred transgenic plants or plant cultivars (i.e. those obtained by genetic engineering) which are to be treated according to the invention include all plants which, as a result of the recombinant modification, received genetic material which imparted particularly advantageous useful properties (“traits”) to these plants. Examples of such properties are better plant growth, increased tolerance to high or low temperatures, increased tolerance to drought or to water or soil salinity, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, tobacco, oilseed rape and also fruit plants (with the fruits apples, pears, citrus fruits and grapes), and particular emphasis is given to maize, soya beans, potatoes, cotton, tobacco and oilseed rape. Traits that are emphasized in particular are increased defence of the plants against insects, arachnids, nematodes, slugs and snails as the result of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits which are also particularly emphasized are the increased defence of plants against fungi, bacteria and viruses by systemic acquired resistance (SAR), systemin, phytoalexins, elicitors and resistance genes and the correspondingly expressed proteins and toxins. Traits that are furthermore particularly emphasized are the increased tolerance of the plants to certain herbicidally active compounds, for example imidazolinones, sulphonylureas, glyphosate or phosphinothricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucoton® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinothricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned also include the varieties sold under the name Clearfield® (for example maize). Of course, these statements also apply to plant cultivars having these genetic traits or genetic traits still to be developed, which cultivars will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The preparation and the use of the active compounds according to the invention is illustrated by the examples below.

### PREPARATION EXAMPLES

### Example 1

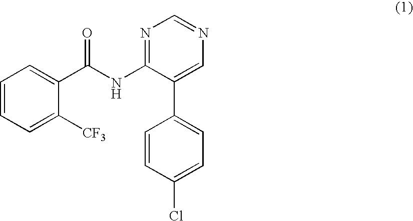

0.2 g (1.0 mmol) of 5-(4-chlorophenyl)-4-pyrimidinylamine is dissolved in 6 ml of tetrahydrofuran, and 0.2 g (2.0 mmol) of triethylamine is added. At 0° C., 0.25 g (1.2 mmol) of 2-trifluoromethylbenzoyl chloride is added. The reaction solution is stirred at 60° C. for 16 h. For work-up, the solution is filtered and concentrated. The crude product is purified by column chromatography (cyclohexane/ethyl acetate 2:1). This gives 0.06 g (15% of theory) of N-[5-(4-chlorophenyl)-4-pyrimidinyl]-2-(trifluoromethyl)benzamide of logP (pH 2.3)=2.57.

### Example 2

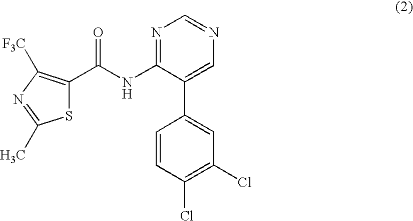

0.21 g (1.0 mmol) of 2-methyl-4-(trifluoromethyl)-1,3-thiazole-5-carboxylic acid, 0.49 g (2.4 mmol) of 1,3-dicyclohexylcarbodiimide and 0.14 g (1.0 mmol) of 1-hydroxybenzotriazole are suspended in 15 ml of dichloromethane. After 5 minutes, 0.24 g (1.0 mmol) of 5-(3,4-dichlorophenyl)-4-pyrimidinylamine is added. The reaction solution is stirred at room temperature for 8 h and then at 40° C. for 16 h. For work-up, the reaction mixture is filtered, washed with saturated sodium bicarbonate solution, dried over sodium sulphate and concentrated. Column chromatography (cyclohexane/ethyl acetate 2:1) gives 0.15 g (34% of theory) of N-[5-(3,4-dichlorophenyl)-4-pyrimidinyl]-2-methyl-4-(trifluoromethyl)-1,3-thiazole-5-carboxamide of logP (pH 2.3)=2.85.

The compounds of the formula (I) listed in Table 1 below can be obtained analogously to Examples 1 and 2 and in accordance with the general descriptions of the processes according to the invention.

TABLE I

| | |
|---|---|
| | (I) |
| 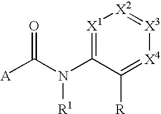 | |

| No. | R | R¹ | X¹ | X² | X³ | X⁴ | A | logP (pH 2.3) |
|---|---|---|---|---|---|---|---|---|
| 3 | 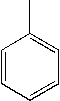 | H | N | CH | N | CH | 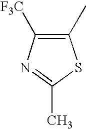 | 2.29 |
| 4 | 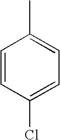 | H | N | CH | N | CH | 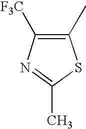 | 2.49 |
| 5 | 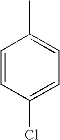 | H | N | CH | N | CH | 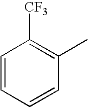 | 1.79 |
| 6 | 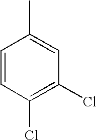 | H | N | CH | N | CH | 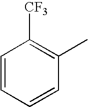 | 2.92 |
| 7 | 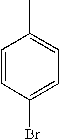 | H | N | CH | N | CH | 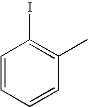 | 2.61 |
| 8 | 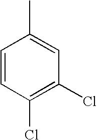 | H | N | CH | N | CH | 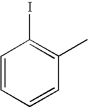 | 2.89 |
| 9 | 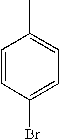 | H | N | CH | N | CH | 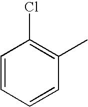 | 2.46 |
| 10 | 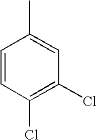 | H | N | CH | N | CH | 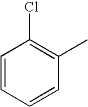 | 2.73 |
| 11 | 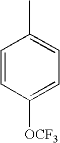 | H | N | CH | N | CN | 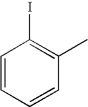 | 2.85 |
| 12 | 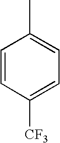 | 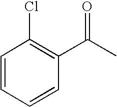 | N | CH | N | CH | 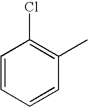 | 4.12 |
| 13 | 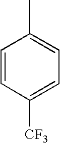 | H | N | CH | N | CH | 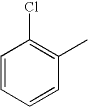 | 2.62 |
| 14 | 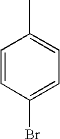 | 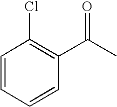 | N | CH | N | CH | 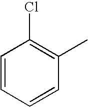 | 2.73 |
| 15 | 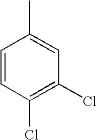 | 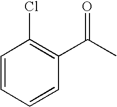 | N | CH | N | CH | 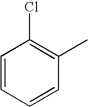 | 2.46 |
| 16 | 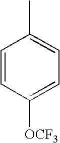 | 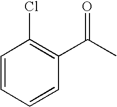 | N | CH | N | CH | 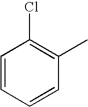 | 4.25 |
| 17 | 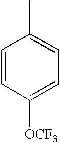 | H | N | CH | N | CH | 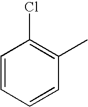 | 2.73 |
| 18 | 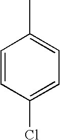 | 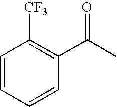 | N | CH | N | CH | 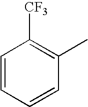 | 4.29 |
| 19 | 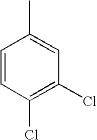 | H | N | CH | N | CH | 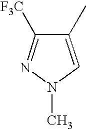 | 2.46 |
| 20 | 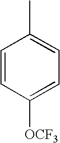 | H | N | CH | N | CH | 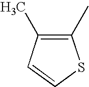 | |

### Preparation of Starting Materials of the Formula (III)

### Example (III-1)

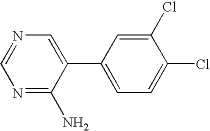

15.0 g (0.08 mol) of 3,4-dichlorobenzyl cyanide, 23.4 g (0.016 mol) of tris(formamino)methane and 1.4 g (0.008 mol) of p-toluenesulphonic acid in 15.5 ml of formamide are stirred at 170-180° C. for 10 h. For work-up, the mixture is acidified with 10% hydrochloric acid, activated charcoal is added and the mixture is stirred for 5 min. After filtration, the filtrate is made basic using 10% strength NaOH. The resulting white precipitate is filtered off with suction, taken up in a mixture of 5% methanol in chloroform and filtered through Celite. This gives 8.2 g (42% of theory) of 5-(3,4-dichlorophenyl)-4-pyrimidinylamine of logP (pH 2.3)=0.87.

The compounds of the formula (III) listed in Table 2 below can be obtained analogously to Example (III-1) and in accordance with the general descriptions of the processes according to the invention.

TABLE 2

| | |
|---|---|
| | (III) |
| 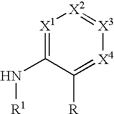 | |

| No. | R | R¹ | X¹ | X² | X³ | X⁴ | logP (pH 2.3) |
|---|---|---|---|---|---|---|---|
| III-2 | 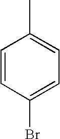 | H | N | CH | N | CH | 0.56 |
| III-3 | 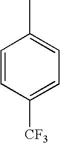 | H | N | CH | N | CH | 0.80 |
| III-4 | 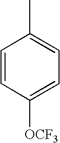 | H | N | CH | N | CH | 1.00 |

The logP values given in the Tables and Preparation Examples above are determined in accordance with EEC Directive 79/831 Annex V.A8 by HPLC (High Performance Liquid Chromatography) on a reverse-phase column (C 18). Temperature: 43° C.

The determination is carried out in the acidic range at pH 2.3 using the mobile phases 0.1% aqueous phosphoric acid and acetonitrile; linear gradient from 10% acetonitrile to 90% acetonitrile.

Calibration is carried out using unbranched alkan-2-ones (having 3 to 16 carbon atoms) with known logP values (determination of the logP values by the retention times using linear interpolation between two successive alkanones).

The lambda max values were determined in the maxima of the chromatographic signals using the UV spectra from 200 nm to 400 nm.

### USE EXAMPLES

### Example A

Sphaerotheca test (cucumber)/protective

| | |
|---|---|
| Solvent: | 49 parts by weight of N,N-dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young cucumber plants are sprayed with the active compound preparation at the stated application rate. One day after the treatment, the plants are inoculated with a spore suspension of Sphaerotheca fuliginea. The plants are then placed in a greenhouse at 70% relative atmospheric humidity and a temperature of 23° C.

Evaluation is carried out 7 days after the inoculation. 0% means an efficacy which corresponds to that of the control, whereas an efficacy of 100% means that no infection is observed.

TABLE A
Sphaerotheca test (cucumber)/protective

| | Application rate of | |
|---|---|---|
| Active compound | active compound in | Efficacy |
| according to the invention | ppm | in % |
| 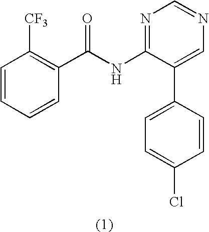 | 500 | 78 |
| 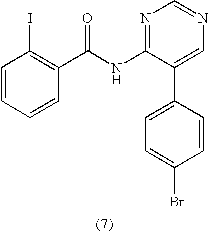 | 500 | 95 |
| 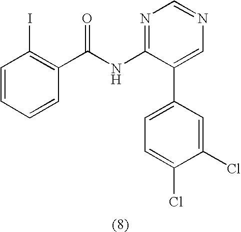 | 500 | 100 |
| 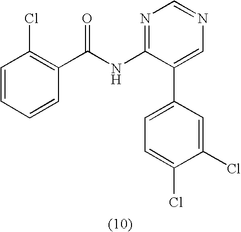 | 500 | 90 |
| 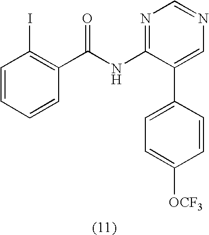 | 500 | 95 |
| 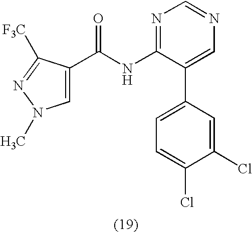 | 500 | 95 |

### Example B

Alternaria test (tomato)/protective

| | |
|---|---|
| Solvent: | 49 parts by weight of N,N-dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young tomato plants are sprayed with the active compound preparation at the stated application rate. One day after the treatment, the plants are inoculated with a spore suspension of Alternaria solani and then remain at 100% relative humidity and 20° C. for 24 h. The plants then remain at 96% relative atmospheric humidity and a temperature of 20° C.

Evaluation is carried out 7 days after the inoculation. 0% means an efficacy which corresponds to that of the control, whereas an efficacy of 100% means that no infection is observed.

TABLE B
Alternaria test (tomato)/protective

| | Application rate of | |
|---|---|---|
| Active compound | active compound in | Efficacy |
| according to the invention | ppm | in % |
| 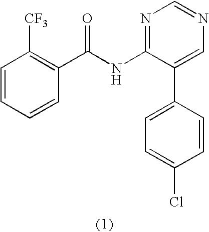 | 500 | 100 |
| 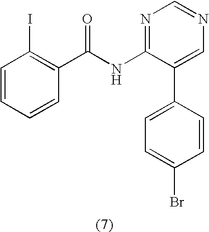 | 500 | 100 |
| 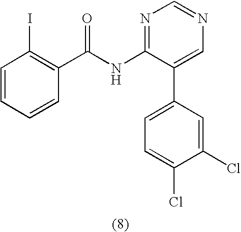 | 500 | 100 |
| 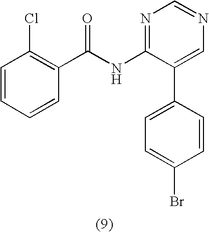 | 500 | 100 |
| 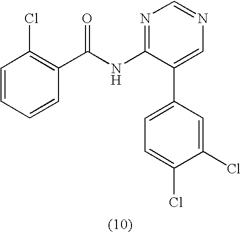 | 500 | 100 |
| 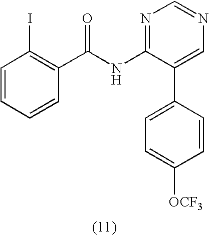 | 500 | 100 |
| 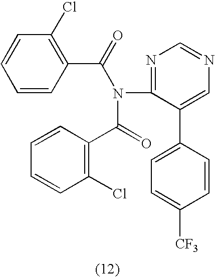 | 500 | 100 |
| 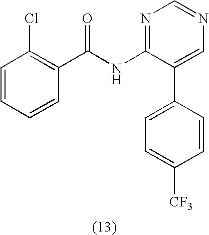 | 500 | 100 |
| 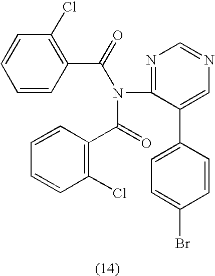 | 500 | 71 |
| 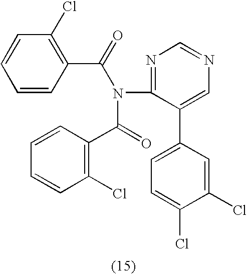 | 500 | 95 |
| 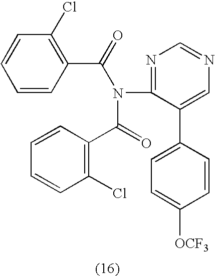 | 500 | 70 |
| 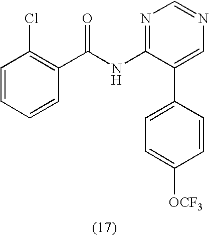 | 500 | 95 |
| 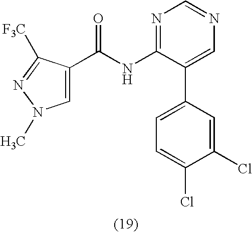 | 500 | 95 |
| 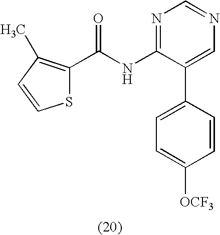 | 500 | 70 |

### Example C

Pyrenophora teres test (barley)/protective

| | |
|---|---|
| Solvent: | 50 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the active compound preparation at the stated application rate. After the spray coating has dried on, the plants are sprayed with a conidia suspension of Pyrenophora teres. The plants remain in an incubation cabin at 20° C. and 100% relative atmospheric humidity for 48 hours. The plants are then placed in a greenhouse at a temperature of about 20° C. and a relative atmospheric humidity of about 80%.

Evaluation is carried out 8 days after the inoculation. 0% means an efficacy which corresponds to that of the control, whereas an efficacy of 100% means that no infection is observed.

TABLE C
Pyrenophora teres test (barley)/protective

| | Application rate of | |
|---|---|---|
| Active compound | active compound in | Efficacy |
| according to the invention | ppm | in % |
| 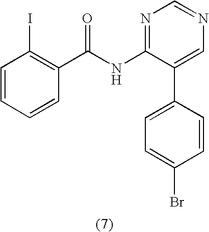 | 1000 | 89 |
| 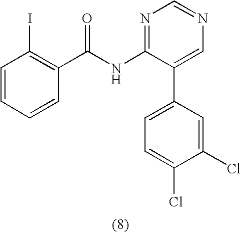 | 1000 | 100 |

### Example D

Podosphaera test (apple)/protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvents and emulsifier, and the concentrate is diluted with water to the desired concentration.

To test for protective activity, young plants are sprayed with the active compound preparation at the stated application rate. After the spray coating has dried on, the plants are inoculated with an aqueous spore suspension of the apple mildew pathogen Podosphaera leucotricha. The plants are then placed in a greenhouse at about 23° C. and a relative atmospheric humidity of about 70%.

Evaluation is carried out 10 days after the inoculation. 0% means an efficacy which corresponds to that of the control, whereas an efficacy of 100% means that no infection is observed.

TABLE D
Podosphaera test (apple)/protective

| | Application rate of | |
|---|---|---|
| Active compound | active compound in | Efficacy |
| according to the invention | ppm | in % |
| 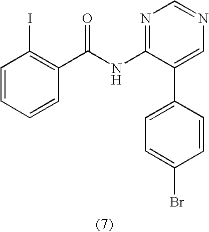 | 100 | 87 |
| 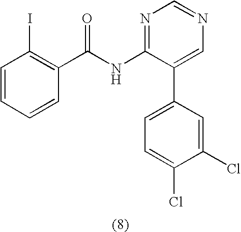 | 100 | 93 |
| 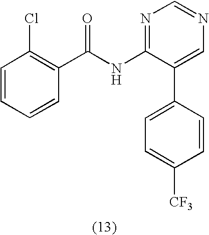 | 100 | 93 |

### Example E

Venturia test (apple)/protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvents and emulsifier, and the concentrate is diluted with water to the desired concentration.

To test for protective activity, young plants are sprayed with the active compound preparation at the stated application rate. After the spray coating has dried on, the plants are inoculated with an aqueous conidia suspension of the apple scab pathogen Venturia inaequalis and then remain in an incubation cabin at about 20° C. and 100% relative atmospheric humidity for 1 day. The plants are then placed in a greenhouse at about 21° C. and a relative atmospheric humidity of about 90%.

Evaluation is carried out 10 days after the inoculation. 0% means an efficacy which corresponds to that of the control, whereas an efficacy of 100% means that no infection is observed.

TABLE E
Venturia test (apple)/protective

| | Application rate of | |
|---|---|---|
| Active compound | active compound in | Efficacy |
| according to the invention | ppm | in % |
| 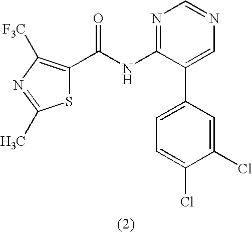 | 100 | 95 |
| 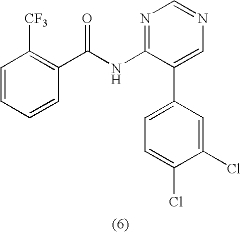 | 100 | 95 |

### Example F

Botrytis test (bean)/protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvents and emulsifier, and the concentrate is diluted with water to the desired concentration.

To test for protective activity, young plants are sprayed with the active compound preparation at the stated application rate. After the spray coating has dried on, two small pieces of agar colonized by Botrytis cinerea are placed onto each leaf. The inoculated plants are placed in a dark chamber at about 20° C. and 100% relative atmospheric humidity.

Two days after the inoculation, the size of the infected areas on the leaves is evaluated. 0% means an efficacy which corresponds to that of the control, whereas an efficacy of 100% means that no infection is observed.

TABLE F
Botrytis test (bean)/protective

| | Application rate of | |
|---|---|---|
| Active compound | active compound in | Efficacy |
| according to the invention | ppm | in % |
| 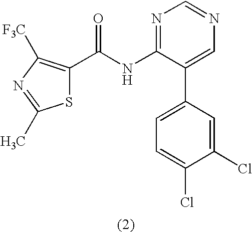 | 500 | 91 |
| 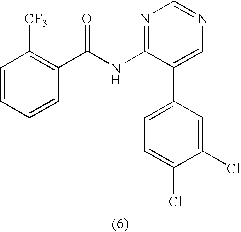 | 500 | 80 |
| 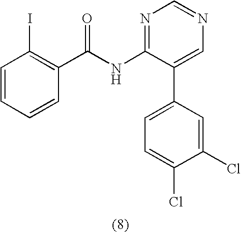 | 500 | 96 |
| 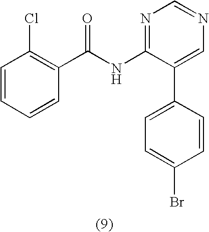 | 500 | 99 |
| 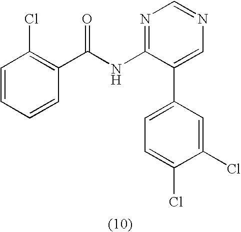 | 500 | 91 |
| 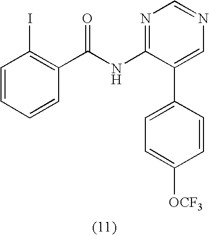 | 500 | 89 |
| 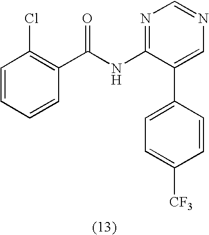 | 500 | 100 |
| 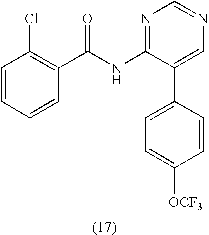 | 500 | 79 |

1. A carboxamide of formula (I) 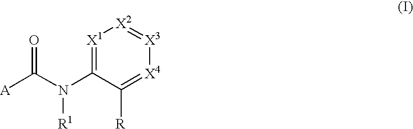
in which
R represents phenyl which is optionally mono- to pentasubstituted by W¹ or represents the grouping 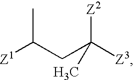
W¹ represents halogen, cyano, nitro, C₁-C₆-alkyl, C₁-C₄-alkoxy, C₁-C₄-alkylthio, C₁-C₄-alkylsulphonyl, C₂-C₆-alkenyl, C₃-C₆-cycloalkyl or represents C₁-C₆-haloalkyl, C₁-C₆-haloalkoxy, C₁-C₆-haloalkylthio or C₁-C₆-haloalkylsulphonyl having in each case 1 to 13 halogen atoms, or represents —C(Q²)=N-Q³ in which
Q² represents hydrogen, hydroxyl, C₁-C₄-alkyl, C₁-C₄-haloalkyl having 1 to 9 halogen atoms or C₃-C₆-cycloalkyl,
Q³ represents hydroxyl, amino, methylamino, phenyl, benzyl or represents in each case optionally halogen-, cyano-, hydroxyl-, C₁-C₄-alkoxy-, C₁-C₄-alkylthio-, C₁-C₄-alkyamino, di(C₁-C₄-alkyl)amino or phenyl-substituted C₁-C₄-alkyl or C₁-C₄-alkoxy, or represents C₂-C₄-alkenyloxy or C₂-C₄-Alkynyloxy,
Z¹ represents hydrogen or methyl,
Z² represents hydrogen or methyl,
Z³ represents methyl or ethyl,
R¹ represents hydrogen, C₁-C₈-alkyl, C₁-C₆-alkylsulphinyl, C₁-C₆-alkylsulphonyl, C₁-C₄-alkoxy-C₁-C₄-alkyl, C₃-C₈-cycloalkyl; C₁-C₆-haloalkyl, C₁-C₄-haloalkylthio, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms;
(C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)-carbonyl, (C₃-C₈-cycloalkyl)carbonyl; (C₁-C₆-haloalkyl)carbonyl, (C₁-C₆-haloalkoxy)carbonyl, (C₁-C₆-haloalkylthio)carbonyl, (halo-C₁-C₄-alkoxy-C₁-C₄-alkyl)carbonyl, (C₃-C₆-haloalkenyloxy)carbonyl, (C₃-C₆-haloalkynyloxy)carbonyl, (C₃-C₈-halocycloalkyl)carbonyl having in each case 1 to 9 fluorine, chlorine and/or bromine atoms; or —CH₂—C≡C—R^{1-A}, —CH₂—CH═CH—R^{1-A}, —CH═C═CH—R^{1-A}, —C(═O)C(═O)R², —CONR³R⁴ or —CH₂NR⁵R⁶,
R^{1-A} represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,
R² represents hydrogen, C₁-C₈-alkyl, C₁-C₈-alkoxy, C₁-C₄-alkoxy-C₁-C₄-alkyl, C₃-C₈-cycloalkyl; C₁-C₆-haloalkyl, C₁-C₆-haloalkoxy, halo-C₁-C₄-alkoxy-C₁-C₄-alkyl, C₃-C₈-halocycloalkyl having in each case 1 to 9 fluorine, chlorine and/or bromine atoms,
R³ and R⁴ independently of one another each represent hydrogen, C₁-C₈-alkyl, C₁-C₄-alkoxy-C₁-C₄-alkyl, C₃-C₈-cycloalkyl; C₁-C₈-haloalkyl, halo-C₁-C₄-alkoxy-C₁-C₄-alkyl, C₃-C₈-halocycloalkyl having in each case 1 to 9 fluorine, chlorine and/or bromine atoms,
R³ and R⁴ furthermore together with the nitrogen atom to which they are attached form a saturated heterocycle which is optionally mono- or polysubstituted by identical or different substituents from a group consisting of halogen and C₁-C₄-alkyl and which has 5 to 8 ring atoms, where the heterocycle may contain 1 or 2 further non-adjacent heteroatoms from the group consisting of oxygen, sulphur and NR⁷,
R⁵ and R⁶ independently of one another represent hydrogen, C₁-C₈-alkyl, C₃-C₈-cycloalkyl; C₁-C₈-haloalkyl, C₃-C₈-halocycloalkyl having in each case 1 to 9 fluorine, chlorine and/or bromine atoms,
R⁵ and R⁶ furthermore together with the nitrogen atom to which they are attached form a saturated heterocycle which is optionally mono- or polysubstituted by identical or different substituents from a group consisting of halogen and C₁-C₄-alkyl and which has 5 to 8 ring atoms, where the heterocycle may contain 1 or 2 further non-adjacent heteroatoms from the group consisting of oxygen, sulphur and NR⁷,
R⁷ represents hydrogen or C₁-C₆-alkyl,
X¹, X², X³ and X⁴ independently of one another represent N or CR⁸, with the proviso that at least one of these radicals represents N,
R⁸ represents hydrogen, fluorine, chlorine, methyl, isopropyl, methylthio or trifluoromethyl,
A represents one of the radicals A1 to A18 below 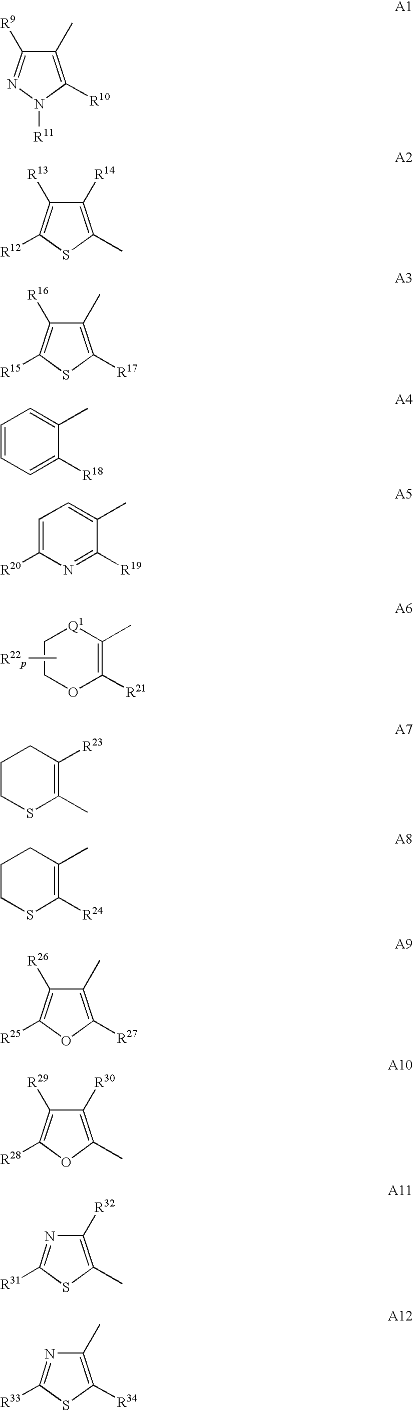 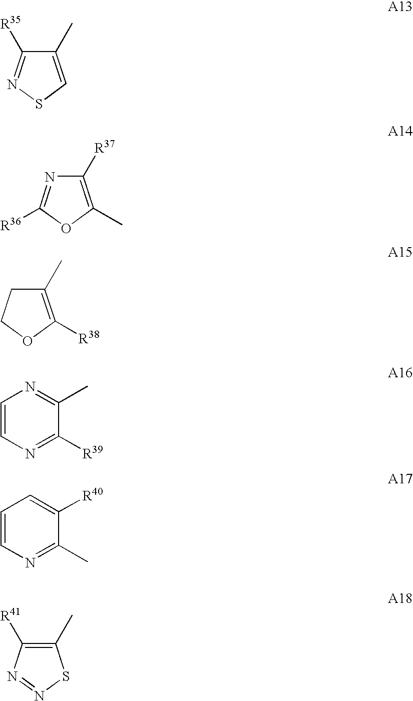
R⁹ represents hydrogen, cyano, halogen, nitro, C₁-C₄-alkyl, C₁-C₄-alkoxy, C₁-C₄-alkylthio, C₃-C₆-cycloalkyl, C₁-C₄-haloalkyl, C₁-C₄-haloalkoxy or C₁-C₄-haloalkylthio having in each case 1 to 5 halogen atoms, aminocarbonyl or aminocarbonyl-C₁-C₄-alkyl,
R¹⁰ represents hydrogen, halogen, cyano, C₁-C₄-alkyl, C₁-C₄-alkoxy or C₁-C₄-alkylthio,
R¹¹ represents hydrogen, C₁-C₄-alkyl, hydroxy-C₁-C₄-alkyl, C₂-C₆-alkenyl, C₃-C₆-cyclo-alkyl, C₁-C₄-alkylthio-C₁-C₄-alkyl, C₁-C₄-alkoxy-C₁-C₄-alkyl, C₁-C₄-haloalkyl, C₁-C₄-haloalkylthio-C₁-C₄-alkyl, C₁-C₄-haloalkoxy-C₁-C₄-alkyl having in each case 1 to 5 halogen atoms, phenyl, (C₁-C₄-alkyl)carbonyl, (C₁-C₄-alkoxy)carbonyl, (C₁-C₄-alkylthio)carbonyl, (C₁-C₄-alkoxy-C₁-C₄-alkyl)carbonyl; (C₁-C₄-haloalkyl)carbonyl, (C₁-C₄-haloalkoxy)carbonyl, (C₁-C₄-haloalkylthio)carbonyl, (halo-C₁-C₄-alkoxy-C₁-C₄-alkyl)carbonyl having in each case 1 to 9 halogen atoms,
R¹² and R¹³ independently of one another represent hydrogen, halogen, C₁-C₄-alkyl or C₁-C₄-haloalkyl having 1 to 5 halogen atoms,
R¹⁴ represents halogen, cyano or C₁-C₄-alkyl, or C₁-C₄-haloalkyl or C₁-C₄-haloalkoxy having in each case 1 to 5 halogen atoms,
R¹⁵ and R¹⁶ independently of one another represent hydrogen, halogen, C₁-C₄-alkyl or C₁-C₄-haloalkyl having 1 to 5 halogen atoms,
R¹⁷ represents hydrogen, C₁-C₄-alkyl or C₁-C₄-haloalkyl having 1 to 5 halogen atoms,
R¹⁸ represents hydrogen, halogen, hydroxyl, cyano, C₁-C₆-alkyl, C₁-C₄-haloalkyl, C₁-C₄-haloalkoxy or C₁-C₄-haloalkylthio having in each case 1 to 5 halogen atoms,
R¹⁹ represents halogen, hydroxyl, cyano, C₁-C₄-alkyl, C₁-C₄-alkoxy, C₁-C₄-alkylthio, C₁-C₄-haloalkyl, C₁-C₄-haloalkylthio or C₁-C₄-haloalkoxy having in each case 1 to 5 halogen atoms,
R²⁰ represents hydrogen, halogen, cyano, C₁-C₄-alkyl, C₁-C₄-alkoxy, C₁-C₄-alkylthio, C₁-C₄-haloalkyl, C₁-C₄-haloalkoxy having in each case 1 to 5 halogen atoms, C₁-C₄-alkylsulphinyl or C₁-C₄-alkylsulphonyl,
R²¹ represents C₁-C₄-alkyl or C₁-C₄-haloalkyl having 1 to 5 halogen atoms,
R²² represents C₁-C₄-alkyl,
Q¹ represents S (sulphur), SO, SO₂ or CH₂,
p represents 0, 1 or 2, where R²² represents identical or different radicals if p represents 2,
R²³ represents C₁-C₄-alkyl or C₁-C₄-haloalkyl having 1 to 5 halogen atoms,
R²⁴ represents C₁-C₄-alkyl or C₁-C₄-haloalkyl having 1 to 5 halogen atoms,
R²⁵ and R²⁶ independently of one another represent hydrogen, halogen, amino, C₄-C₄-alkyl or C₁-C₄-haloalkyl having 1 to 5 halogen atoms,
R²⁷ represents hydrogen, C₁-C₄-alkyl or C₁-C₄-haloalkyl having 1 to 5 halogen atoms,
R²⁸ and R²⁹ independently of one another represent hydrogen, halogen, amino, nitro, C₁-C₄-alkyl or C₁-C₄-haloalkyl having 1 to 5 halogen atoms,
R³⁰ represents hydrogen, halogen, C₁-C₄-alkyl or C₁-C₄-haloalkyl having 1 to 5 halogen atoms,
R³¹ represents hydrogen, halogen, amino, C₁-C₄-alkylamino, di-(C₁-C₄-alkyl)amino, cyano, C₁-C₄-alkyl or C₁-C₄-haloalkyl having 1 to 5 halogen atoms,
R³² represents halogen, C₁-C₄-alkyl or C₁-C₄-haloalkyl having 1 to 5 halogen atoms,
R³³ represents hydrogen, halogen, amino, C₁-C₄-alkylamino, di-(C₁-C₄-alkyl)amino, cyano, C₁-C₄-alkyl or C₁-C₄-haloalkyl having 1 to 5 halogen atoms,
R³⁴ represents halogen, C₂-C₄-alkyl or C₁-C₄-haloalkyl having 1 to 5 halogen atoms,
R³⁵ represents halogen, C₁-C₄-alkyl or C₁-C₄-haloalkyl having 1 to 5 halogen atoms,
R³⁶ represents hydrogen or C₁-C₄-alkyl,
R³⁷ represents halogen or C₁-C₄-alkyl,
R³⁸ represents C₁-C₄-alkyl or C₁-C₄-haloalkyl having 1 to 5 halogen atoms,
R³⁹ represents hydrogen, halogen, C₁-C₄-alkyl or C₁-C₄-haloalkyl having 1 to 5 halogen atoms,
R⁴⁰ represents halogen, hydroxyl, C₁-C₄-alkyl, C₁-C₄-alkoxy, C₁-C₄-alkylthio, C₁-C₄-haloalkyl, C₁-C₄-halo alkylthio or C₁-C₄-haloalkoxy having in each case 1 to 5 halogen atoms,
R⁴¹ represents C₁-C₄-alkyl.

2. A process for preparing a carboxamide according to claim 1, comprising reacting
(a) a carbonyl halide of formula (II) 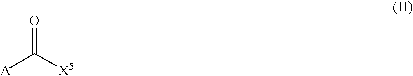
in which
X⁵ represents halogen or hydroxyl,
with an aniline derivative of formula (III) 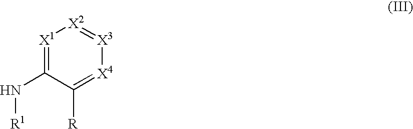
if appropriate in the presence of a coupling agent, if appropriate in the presence of an acid binder and if appropriate in the presence of a diluent,
or
(b) reacting a carboxamide of formula (I-a) 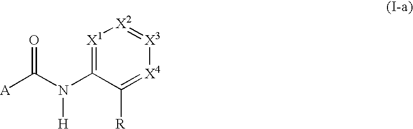
with a halide of formula (IV)
R^{1-B}-Hal (IV)
in which
R^{1-B} represents C₁-C₈-alkyl, C₁-C₆-alkylsulphinyl, C₁-C₆-alkylsulphonyl, C₁-C₄-alkoxy-C₁-C₄-alkyl, C₃-C₈-cycloalkyl; C₁-C₆-haloalkyl, C₁-C₄-haloalkylthio, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo(C₁-C₃-alkyl)carbonyl-C₁-C₃-alkyl, halo(C₁-C₃-alkoxy)carbonyl-C₁-C₃-alkyl having in each case 1 to 13 fluorine, chlorine and/or bromine atoms;
(C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkoxy)carbonyl, (C₁-C₆-haloalkylthio)carbonyl, (halo-C₁-C₄-alkoxy-C₁-C₄-alkyl)carbonyl, (C₃-C₆-haloalkenyloxy)carbonyl, (C₃-C₆-haloalkynyloxy)carbonyl, (C₃-C₈-halocycloalkyl)carbonyl having in each case 1 to 9 fluorine, chlorine and/or bromine atoms; or —CH₂—C≡C—R^{1-A}, —CH₂—CH═CH—R^{1-A}, —CH═C═CH—R^{1-A}, —C(═O)C(═O)R², —CONR³R⁴ or —CH₂NR⁵R⁶,
Hal represents chlorine, bromine or iodine,
in the presence of a base and in the presence of a diluent.

3. A composition for controlling an unwanted microorganism, comprising at least one carboxamide according to claim 1, and an extender and/or surfactant.

4. A carboxamide according to claim 1 used for controlling an unwanted microorganism.

5. A method for controlling an unwanted microorganism, comprising applying a carboxamide according to claim 1 to the microorganism and/or a habitat thereof.

6. A process for preparing a composition for controlling an unwanted microorganism, comprising mixing a carboxamide according to claim 1 with an extender and/or surfactants.

7. A carboxamide according to claim 1 for treating seed.

8. A carboxamide according to claim 1 for treating a transgenic plant.

9. A carboxamide according to claim 1 for treating seed of a transgenic plant.

10. An aniline derivative of formula (III-a) 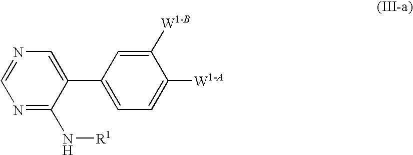
in which
W^{1-A} and W^{1-B} either both represent chlorine
or
W^{1-A} represents trifluoromethyl or trifluoromethoxy and W^{1-B} represents hydrogen.
